(19) United States
(12) Patent Application Publication
(10) Pub. No.: US 2017/0234880 A1
(43) Pub. Date: Aug. 17, 2017

(54) DIAGNOSIS OF CANCER

(71) Applicant: Agency for Science, Technology and Research, Singapore (SG)

(72) Inventor: Qi Zeng, Singapore (SG)

(73) Assignee: Agency for Science, Technology and Research, Singapore (SG)

(21) Appl. No.: 15/503,155

(22) PCT Filed: Aug. 13, 2015
(86) PCT No.: PCT/SG2015/050259

§ 371 Date: Feb. 10, 2017

(30) Foreign Application Priority Data
Aug. 13, 2014 (SG) 10201404895X

(51) Int. Cl. G01N 33/574; C07K 16/40

(52) U.S. Cl. CPC G01N 33/5748; C07K 16/40; C12Y 301/03048; G01N 2333/82

(57) ABSTRACT

The data provided herewith show that, biomarkers found within the cells of a tumor or cancer may also be present at adequate levels within the patient's urine to enable detection. Method of diagnosis, patient selection and treatment are provided, along with kits and devices.

### FIELD OF THE INVENTION

The present invention relates to the fields of cell biology, molecular biology and biochemistry. This invention also relates to the field of medicine. In particular, it relates to diagnosis of diseases, in particular cancer.

### BACKGROUND TO THE INVENTION

Cancer has been the subject of intense investigation in the past two decades. However, the underlying causes responsible for cancer metastasis are poorly understood and most types of cancer prevention are still limited.

Antibody based therapy has proven to be effective for cancer treatment; however, this approach has traditionally been limited to extracellular or secreted proteins expressed by cancer cells. Thus, a number of potential cancer or tumour markers and cancer antigens have been identified in the literature and antibody therapies have been developed against some of them.

For example, the well-known cancer therapy Herceptin (Trastuzumab) is a monoclonal antibody that can kill HER2-positive cancer cells. Herceptin binds to the HER2 (human epidermal growth factor receptor 2) antigen on the cancer cell. Likewise, Bevacizumab (Avastin™) is a monoclonal antibody targeted against vascular endothelial growth factor (VEGF), one of the growth factors implicated in the formation of new blood vessels. By inhibiting angiogenesis, Bevacizumab prevents tumour cells from receiving a constant supply of blood to receive the oxygen and nutrients the tumour needs to survive.

However, the applicability of antibody therapeutics for different cancers is not universal. One of the limitations that has prevented the general use of antibody therapeutics is the large size of antibody molecules and their consequent inability to cross the plasma or cell membrane. In the absence of modification, antibodies (including monoclonal antibodies) are only generally suitable for targeting cancer antigens located at the surface or exterior of host cells. In the examples above, HER2 receptor is located on the cell surface and is hence accessible for antibody binding by Herceptin. Likewise, VEGF is secreted into the bloodstream and is able to be bound by Bevacizumab.

Most oncogenic proteins are intracellular proteins (such as intracellular phosphatases, intracellular kinases, transcription factors, etc), and have remained under-explored by the approach of antibody therapies. The long held view that antibodies are too large to penetrate cell membrane has hampered the technology of antibody therapy used in targeting intracellular proteins.

We previously showed that three different antibodies could target three intracellular proteins respectively: PRL-3 (phosphatase of regenerating liver 3), a cancer-associated phosphatase; EGFP (enhanced green fluorescent protein), a general reporter; and mT (polyomavirus middle T), the polyomavirus middle T oncoprotein (WO2011/065923). Only PRL-3 intracellular phosphatase (an enzyme) has been linked to human cancer metastasis (see Saha et al., Science 294; 1343 (2001) and Wang et al., Cancer cell 18; 52-63 (2010)), the other two intracellular proteins (EGFP and middle T) were used to elucidate the general phenomena that antibodies can target intracellular proteins.

### SUMMARY OF THE INVENTION

The inventors have uncovered the presence of several oncoproteins preferentially in the urine samples or urine exosomes of cancer patients as compared to those of normal controls. In this study, we tested several intracellular and extracellular proteins: PRL-3, PRL-1, VHZ, myc, ras, actin, GAPDH, p53, N-Cadherin, Her2, EGFR, FLT, Estrogen

Receptor, PDGF, PDGF-a. Analysis was performed with western blot. Hundreds of cancer urine samples have been examined, including bladder cancer, lung cancer, lymphoma, prostatic adenocarcinoma, breast cancer, stomach cancer or gastric cancer, nasopharynx cancer and nasopharyngeal carcinoma (NPC) and compared with control normal urine samples. The levels of these oncoproteins in the urine are associated with cancer stages such as initial and later stages of bladder cancer. These discoveries offer a simple test for cancer, by determining the presence of urinary oncoprotein using various immunological assays. Our data show that intracellular oncoproteins can be unconventionally secreted out through exosomes to travel long distance within body fluid (blood, urine, saliva, etc). Disclosed herein are urine tests to detect the presence of oncoproteins for diagnostics, cancer staging, cancer therapy, and monitoring anti-cancer therapy.

The data provided herewith show that, surprisingly, biomarkers found within the cells of a tumor or cancer may be present at adequate levels within the patient's urine to enable detection. Moreover, the inventors have surprisingly found that certain oncoproteins may be detected in urine at a very early stage in the development of the cancer. It is surprising that sufficient amounts of oncoprotein to enable detection and diagnosis are present distant from the tumor, in the patients urine, during these initial stages.

We found secreted PRL3 oncoprotein in 62% of multiple types of human cancer urine and in 100% of cancer urine derived from PRL3+ (but not PRL3−) tumor bearing mice. Urinary PRL3 levels were significantly reduced after effective treatment with anti-PRL3 antibody. Urinary PRL3 is therefore proposed as a novel diagnostic and surrogate biomarker for therapeutic response monitoring of anti-PRL3 antibody therapy in cancer.

Using bodily fluid samples such as urine may be particularly advantageous where the cancer is inaccessible.

The invention provides a method for determining whether an individual does, or does not, have cancer, the method comprises determining the presence or absence of an oncoprotein in a sample of a bodily fluid from the individual. The presence of the oncoprotein may be indicative that the individual has cancer. In some cases, the amount of oncoprotein is quantified. The amount of oncoprotein present in the sample may be indicative that the individual has cancer. For example, a higher level of oncoprotein may indicate the presence of a cancer. The amount of oncoprotein may indicate the stage of the cancer, such as early, mid or late stage. The method may involve the detection of exosomal oncoprotein. That is, the detection of oncoprotein within or attached to, exosomes, within the bodily fluid.

The methods disclosed herein may be used to monitor the response of a patient to a therapeutic treatment. For example, the presence or level of oncoprotein in urine may be determined before and after a therapeutic treatment is administered. The therapeutic treatment may be chemotherapy, radiotherapy or other therapeutic treatment. A reduction in the level of oncoprotein may indicate that the therapy is having an anti-cancer effect.

The methods disclosed herein may be used to select a patient for treatment with anti-cancer therapy, or to determine that a patient is suitable for such treatment. In some cases, methods disclosed herein relate to the treatment of patients, determined to be suitable for treatment by the methods disclosed herein. Agents for use in such methods are also disclosed.

Samples of bodily fluid useful in the methods of the invention include urine, saliva, blood or plasma, or any other bodily fluid, including breast milk. Preferably, the bodily fluid is urine.

The oncoprotein may be present in exosomes in the sample or may be soluble. The method may involve the isolation of exosomes from the sample, and determining the presence or absence of an oncoprotein in the exosomes. The method may involve a step of concentrating the exosomes, relative to the sample isolated from the individual. The sample of exosomes may or may not be treated to release the contents of the exosomes, such as lysing or bursting the exosomes. The method may be performed on a sample of exosomes that has been previously isolated from a sample of bodily fluid. In particular, the presence of oncoprotein in exosomes in the sample may indicate that the patient has bladder cancer. In some cases, the oncoprotein is soluble, such as soluble PRL3. Soluble oncoprotein may not be associated with, or present in, exosomes in the sample.

The presence or absence of the oncoprotein may involve an immunoassay, such as an ELISA or western blot based method. The method may involve the use of antibodies or aptamers. The antibodies may be any antibody that is capable of binding specifically to the oncoprotein. Antibodies may be monoclonal or polyclonal, they may be human, rabbit or mouse antibodies, or from any suitable mammal, or may be produced in cell culture. The antibodies may be humanised or chimeric. The antibody may be Antibody 223 or 318 as discussed in Li et al (Clin. Canc. Res. (2005) 11:2195-204). The antibody may bind to a region of an oncoprotein that is normally presented within the cell, such as within the plasma membrane, cytoplasm or nucleus. In some cases, the antibody does not bind to an extracellular portion of an oncoprotein, such as a portion of the oncoprotein normally presented on the surface of a cell.

Oncoproteins useful in the methods disclosed herein are upregulated or overexpressed in cancers as compared to non-cancerous tissues. In some cases the oncoprotein carries a mutation as compared to the protein that is expressed in non-cancerous tissue. Oncoproteins as discussed herein may be intracellular or extracellular. That is to say that their expression is substantially within the cell, such as in the nucleus, cytoplasm, or internal side of the cell membrane, or substantially on the outside of the cell, such as on the cell surface. Preferably the oncoprotein is an intracellular oncoprotein.

In preferred methods described herein, the oncoprotein is selected from PRL3, PRL1, VHZ, c-myc, H-ras, AKT-1, p53, Rac1, FAK, Runx1, Estrogen Receptor (ER), PTEN, b-actin or GAPDH. The p53 may be mutant p53. For example, the p53 protein may have a proline residue instead of an arginine residue at position 72. Preferably the oncoprotein is PRL3, PRL1 or VHZ. In some cases the oncoprotein is a form of GAPDH or b-Actin which is mutated relative to the form found in non-cancerous tissues. In some cases, the presence or absence of more than one oncoprotein is determined. The oncoprotein may be Her2, N-Cadherin, PDGF receptor (alpha), FLT-3 or p-EGFR.

Cancers detectable by the methods disclosed herein include gastric cancer, bladder cancer, lung cancer, breast cancer, stomach cancer, nasopharyngeal cancer, prostate cancer (such as prostatic adenocarcinoma or prostatic hyperplasia, particularly being prostatic hyperplasia). The cancer may be distant from the source of the sample. The cancer may be one that is difficult and/or invasive to access for example to sample or biopsy.

In some cancers the methods involve determining whether the level of oncoprotein in the sample is higher or lower than the level in a sample obtained from an individual who does not have cancer. In some cases, a higher level of oncoprotein relative to a non-cancerous individual is indicative that the individual has cancer. In some cases, the level of expression is indicative of the stage of the cancer. A high level of expression may indicate that the individual has a late stage, or advanced cancer.

The individual is preferably a human individual, and the oncoprotein detected is preferably a human oncoprotein.

In some cases, the oncoprotein is not Her2, N-Cadherin, PDGF receptor (alpha), FLT-3 or p-EGFR. In some cases, the cancer is not bladder cancer.

In some cases the methods of the invention involve determining the stage of the cancer in the individual. That is, the presence or absence of certain oncoproteins in the sample may be indicative that the cancer is early, mid, or late stage. The presence or absence of a certain oncoprotein may be indicative that the cancer is, or will be metastatic. In some cases, the amount of oncoprotein is indicative of the stage of the cancer.

The methods may involve a comparison step in which the level of biomarker in a sample from an individual is compared to the level of the biomarker in a sample obtained for a non-cancerous individual. The level may be compared directly with a sample from the non-cancerous individual (e.g. a control sample), or may be compared with a database of one or more reference values.

Bodily fluid samples may be obtained from an individual by any suitable method known in the art. The sample may have been obtained prior to commencing the methods disclosed herein, or may be obtained as part of that method. The bodily fluid may be a fluid in which exosomes are present. The method may involve the detection of oncoproteins in the sample such as soluble oncoprotein. The method may involve the isolation of exosomes from the sample, or concentration of exosomes within the sample, and the detection of oncoproteins in the exosomes.

Methods for isolating exosomes from samples are known in the art. For example, the EXOQUICK-TC™ process is a commercially available technique in which a sample is incubated in a precipitation solution overnight prior to centrifugation to form a solid precipitate containing exosomes. Other methods, for isolating or concentrating exosomes include ultra-configuration.

Exosomes are cell derived vesicles. They may have a diameter of 30-100 nm. They are membrane bound, and released from the plasma membrane. Exosomes contain various molecular constituents, including miRNA and protein. There is increasing clinical interest in exosomes, particularly in their use in diagnosis, prognosis and therapy. Exosomes have been identified in a variety of biological fluids, including urine, blood, serume, saliva and breast milk.

Exosomes isolated by methods of the invention may be ruptured prior to the detection of oncoproteins. The methods disclosed herein may additionally involve the detection of exosomal markers, such as CD63, or other proteins or RNA sequences that are characteristic of exosomes.

Also disclosed herein are kits for determining the presence or absence of an oncoprotein in a sample. The kit may comprise an antibody that is specific to an oncoprotein.

Also provided herein is a solid support on which an anti-oncoprotein antibody is bound. The solid support is thus suitable for binding to oncoprotein in a sample, such that oncoprotein can be partitioned from the sample, and the detected.

Also disclosed herein is a lateral flow test, or lateral flow immunochromatographic assay (“lateral flow test”) for detecting the presence or absence of a target in a sample. The test may be suitable for home testing, point of care testing or laboratory use. The lateral flow test may contain one or more anti-oncoprotein antibodies.

The kit may include one or more solutions for preparing a biological fluid for detection of an oncoprotein, and/or for washing oncoprotein bound to the support and/or detecting bound oncoprotein. The kit may include a lateral flow test.

Any method suitable for the detection of a protein in a sample may be used in the methods described herein. The method may involve the detection of the presence or absence of the oncoprotein. The method may involve the quantification of the level of expression of the oncoprotein.

In some cases, the level of oncoprotein is indicative of the stage of the cancer. Thus a high level of expression of a certain oncoprotein may be indicative that the cancer is at a more advanced stage, such as a late stage cancer. In some cases, the presence of the oncoprotein may be indicative of a late stage cancer.

Methods useful herein include western blot or dot blot. Such methods involve the detection of a particular oncoprotein by exposing the sample to an antibody or other agent capable of specifically binding to the oncoprotein.

Particularly preferred methods involve Enzyme-linked immunosorbent assays (ELISA), such as sandwich ELISA. ELISA involves the use of a captive agent to specifically bind an oncoprotein to a surface, and a detecting agent is applied to detect bound oncoprotein. Typically, the capture and detecting agents are antibodies. The detecting agent may be linked to a reporter, preferably an enzyme. Substrate is then applied to the surface, and bound oncoproteins detected throughout the reaction of the substrate with the detecting agent.

In some cases, the ELISA may be performed using a solid support which is immersible in the biological fluid, or in the sample of exosomes obtained from a biological fluid. The solid support may be a plate, dipstick or bead, or other support known in the art.

Also included herein is a kit for detecting or measuring oncoprotein in a bodily fluid. The kit may include a lateral flow test device. The kit may include instructions for use. The lateral flow test may include a reaction zone, a test zone, and optionally a control zone.

Suitable antibodies for use in an ELISA based method for detecting an oncoprotein include any anti-oncoprotein antibody known in the art. For detecting PRL3, the antibody may be monoclonal antibodies 223 or 318 as disclosed in Li et al, or polyclonal antibody serum from rabbit #1992 or #1993. Any combination of antibodies may be used as capture or detection agent. Preferably however the capture and detection agents are different. In the case of PRL3, a particularly preferred combination is the use of monoclonal antibody 223 as capture antibody. Rabbit antiserum #1992 is preferred as detector. The detector may be linked to gold particles to allow for detection.

Methods according to the invention may include the following steps:
a) providing a biological sample obtained from an individual;
b) exposing the biological sample to a solid support on which an oncoprotein binding agent is immobilised, such that oncoprotein within the biological sample is immobilised to the solid support;
c) partitioning the oncoprotein from the biological sample;
d) detecting the oncoprotein.

The presence of the oncoprotein may be indicative that the individual has cancer. The method may involve quantifying the amount of oncoprotein detected. The method may involve one or more washing steps, such as following immobilisation of the oncoprotein on the solid support. The method may involve a comparison step in which the result obtained in step (d) is compared to the results obtained from an individual known to not have cancer. The comparison may involve comparison with known values in a database.

In some cases, the solid support is a nitrocellulose membrane, particularly a nitrocellulose AE99. Preferably a monoclonal antibody is bound to the solid support as the capture antibody. Preferably the antibody is antibody 223. The assay may involve exposing the biological sample to the capture antibody for 10-20 minutes, preferably 11-19 minutes, preferably 14-16 minutes, preferably around 15 minutes.

Preferably a polyclonal antibody is used as the detector agent. Preferably rabbit anti-PRL3#1992 serum is used as the detector agent. Preferably the detector agent is conjugated to gold particles to allow for detection.

Following diagnosis of a cancer due to the presence or level of a particular oncoprotein, the individual may be prescribed or undergo a relevant therapeutic treatment. For example, the detection of a particular oncoprotein may be indicative that a particular immunotherapy may be beneficial. For example, identification of the presence of PRL3 may be indicative that the patient would benefit from PRL3 immunotherapy, such as anti-PRL3 antibody therapy.

### DETAILED DESCRIPTION

We describe various immunological assays such as western blot, dot blot, ELISA. We also describe pregnancy-like test to detect oncoproteins in urines and blood samples (and also saliva).

We investigate whether the levels of oncoproteins are associated with stages of cancers.

Using urine and blood samples to screen biomarkers is easier compared with using tumor samples since tumors can be difficult to be reached, such as brain tumors in internal center position.

We have 2 independent PRL-3 monoclonal antibodies (#318 or #223); we also have polyclonal rabbit antibodies that react with PRL-3 and its related PRL-1 and PRL-2. In addition to western blot and Elisa assays, we can make paper strips that contain either PRL-3 clonal antibody #318 or #223 for capturing PRL-3 antigen from urine or sera (perhaps saliva) samples, a secondary antibody conjugated with HRP or AP will clearly reveal color of antibody-antigen enzyme reaction on the strips or plate reflecting the PRL-3 antigen is present. This technology is similar to a home pregnancy test (reaction zone, test zone, control zone), which can be applied to any other biomarkers detected in urine samples.

We also use similar technologies to test other secreted oncotargets in simple urine samples.

That intracellular proteins can be excreted out via exosome to urines has not previously been reported. This is the first study to demonstrate this and provides simple ways to detect cancers and to reflect stages of cancers. Disclosed herein is the data obtained by analyzing cancer urine samples or the corresponding exosomes purified from hundreds urine samples since last year.

As used herein, the term “biomarker” refers to a protein indicative a biological condition or disease in an individual, such as cancer. Biomarkers of the invention are typically proteins, or oncoproteins. Oncoproteins are proteins with the potential to cause cancer, or associated with cancer. In particular, the invention relates to biomarkers which are intracellular oncoproteins.

Intracellular Oncoproteins

The inventors have determined that intracellular oncoproteins may be unconventionally secreted out through exosomes to travel long distances within the body. The present invention therefore particularly relates to intracellular oncoproteins.

Intracellular oncoproteins and intracellular antigens are known in the art and may include, amongst others, any one or more of the following, or their variants, derivatives, homologues or fragments.

Suitable intracellular oncoproteins will be appreciable by the skilled person. Genes specifically up-regulated during tumor formation but poorly or not expressed in host tissues are particularly promising as tumor-specific targets. For cancers that show a genetic link, immunization of immune-competent young susceptible family members with an antigen (epitope-based peptide vaccine) that is associated with the familial cancer could prime the immune system against that oncoprotein. These endogenously stimulated antibodies could then potentially combat cancer cells expressing that particular oncoprotein. The results described herein suggest that antibody-based therapy and vaccination against cancer may be extended to a wider variety of intracellular oncoproteins as therapeutic targets. The whole class of intracellular oncoproteins previously thought to be un-targetable by therapeutic antibodies or vaccinations can now expand the scope for tailor-made cancer therapies as well as usher in a new era of cancer vaccines. We expect that one potential advantage of using intracellular self-antigens is that they may have a better chance of provoking an immune response than extracellular self-antigens because immune cells targeting extracellular self-antigens are generally eliminated during development. We found that compared to exogenously delivered antibodies, antigen-induced antibody therapy could achieve similar antitumor therapeutic efficacy. Because existing conventional clinical antibody therapy is costly, vaccination may be more useful and economical as a means of inducing high titers of antigen-induced antibodies. This concept of “cancer vaccination” is promising and challenging.

Oncoproteins as described herein are proteins involved in the regulation or synthesis of proteins linked to tumorigenic cell growth. Oncoproteins may be oncogenic polypeptides, involved in the transformation of normal cells into cancer cells. Oncoproteins may have higher expression in tumor cells than in normal cells. The oncoproteins are intracellular, meaning that they are located inside the cell, for example in the nucleus or cytoplasm, or attached to the intracellular surface of the cell membrane. Preferably, the oncoproteins are self-antigens, meaning that they are proteins normally found in the animal, and form part of the protein population expressed from the genome of the animal, and are not heterologous to that animal, such as viral proteins.

Alternatively, the intracellular oncoprotein may be an oncoprotein that has an intracellular region. For example it may be a membrane anchored protein that has a region which extends into the cytoplasm.

Oncoprotein can be a non-self-antigen, such as viral protein expressed by infected cells. In some cases the intracellular oncoprotein is not derived from a microorganism. For example, is not a viral oncoprotein, or is not a bacterial oncoprotein, or is not a fungal oncoprotein. Preferably, the oncoprotein is a self-antigen. One potential advantage of using intracellular self-antigens is that they may have a better chance of provoking an immune response than extracellular self-antigens because immune cells targeting extracellular self-antigens are generally eliminated during development.

PRL-3

The following text is adapted from OMIM entry 606449.

PRL-3 is also known as Protein-Tyrosine Phosphatase, Type 4A, 3; PTP4A3. The chromosomal location of PRL-3 is at gene map locus 8q24.3.

In the heart, protein kinases regulate contractility, ion transport, metabolism, and gene expression. Phosphatases, in addition to their role in dephosphorylation, are involved in cardiac hypertrophy and dysfunction.

By database searching and screening of a heart cDNA library, Matter et al. 2001, Biochem. Biophys. Res. Commun. 283: 1061-1068 identified a cDNA encoding PTP4A3, which they termed PRL3. The deduced PRL3 protein is 76% identical to PRL1 (PTP4A1 ; 601585) and 96% identical to mouse Prl3. Northern blot analysis revealed expression of an approximately 2.3-kb PRL3 transcript predominantly in heart and skeletal muscle, with lower expression in pancreas. This expression pattern is distinct from the wider expression of PRL1 and PRL2 (PTP4A2; 601584). In situ hybridization analysis localized PRL3 expression to cardiomyocytes. Tris glycine gel analysis showed that PRL3 is expressed as a 22-kD protein. Functional and mutation analyses indicated that phosphate cleavage is dependent on cysl 04 of PRL3. Overexpression of PRL3 resulted in increased cell growth. Western blot analysis showed dephosphorylation of p130cas (BCAR1 ; 602941) in response to angiotensin 11 (106150), suggesting a role for PRL3 in the modulation of intracellular calcium transients induced by angiotensin II.

To gain insights into the molecular basis for metastasis, Saha et al. 2001, Science 294: 1343-1346 compared the global gene expression profile of metastatic colorectal cancer with that of primary cancers, benign colorectal tumors, and normal colorectal epithelium. PRL3 was expressed at high levels in each of 18 cancer metastases studied but at lower levels in nonmetastatic tumors and normal colorectal epithelium. In 3 of 12 metastases examined, multiple copies of the PRL3 gene were found within a small amplicon located at chromosome 8q24.3. Saha et al. (2001) concluded that the PRL3 gene is important for colorectal cancer metastasis.

Using the Stanford G3 radiation hybrid panel and database sequence analysis, Saha et al. (2001) mapped the PRL3 gene to surrounding marker 145.20. The PRL3 gene is also tightly linked to marker SHGC-22154, which is located at 8q24.3, approximately 3 Mb from the 8q telomere. Mouse and human PRL-3 proteins were described in detail in Li et al (2005), Clin Cancer Res; I 1:2195-204.

PRL-3 Sequences

The methods and compositions described here make use of PRL-3 polypeptides, which are described in detail below. As used here, the term “PRL-3” is intended to refer to a sequence selected from the following.

| Unigene | Version | Description |
|---|---|---|
| AF041434.1 | GI: 3406429 | Homo sapiens potentially prenylated protein |
| | | tyrosine phosphatase hPRL-3 mRNA, complete cds |
| BT007303.1 | GI: 30583444 | Homo sapiens protein tyrosine phosphatase type |
| | | IVA, member 3 mRNA, complete cds |
| AK128380.1 | GI: 34535719 | Homo sapiens cDNA FLJ46523 fis, clone |
| | | THYMU3034099 |
| NM_007079.2 | GI: 14589853 | Homo sapiens protein tyrosine phosphatase type |
| | | IVA, member 3 (PTP4A3), transcript variant 2, |
| | | mRNA |
| AY819648.1 | GI: 55977462 | Homo sapiens HCV p7-transregulated protein 2 |
| | | mRNA, complete cds |
| BC003105.1 | GI: 13111874 | Homo sapiens protein tyrosine phosphatase type |
| | | IVA, member 3, mRNA (cDNA clone MGC: 1950 |
| | | IMAGE: 3357244), complete cds |
| NM_032611.1 | GI: 14589855 | Homo sapiens protein tyrosine phosphatase type |
| | | IVA, member 3 (PTP4A3), transcript variant 1, |
| | | mRNA |
| AK311257.1 | GI: 164696021 | Homo sapiens cDNA, FLJ 18299 |
| U87168.1 | GI: 1842085 | Human protein tyrosine phosphatase homolog |
| | | hPRL-R mRNA, partial cds |
| BC066043.1 | GI: 42406367 | Mus musculus protein tyrosine phosphatase 4a3, |
| | | mRNA (cDNA clone MGC: 90066 IMAGE: 6415021), |
| | | complete cds |
| AJ276554.1 | GI: 26985935 | Homo sapiens mRNA for protein tyrosine |
| | | phosphatase hPRL-3, short form |
| AK190358.1 | GI: 56014535 | Mus musculus cDNA, clone: YIG0102103, |
| | | strand: plus, reference: ENSEMBL:Mouse- |
| | | Transcript-ENST: ENSMUST00000053232, based |
| | | on BLAT search |
| CT010215.1 | GI: 71059758 | Mus musculus full open reading frame cDNA clone |
| | | RZPDo836H0950D for gene Ptp4a3, Protein |
| | | tyrosine phosphatase 4a3; complete cds, incl. |
| | | stopcodon |
| AK147489.1 | GI: 74184679 | Mus musculus adult male brain UNDEFINED CELL |
| | | LINE cDNA, RIKEN full-length enriched library, |
| | | clone: M5C1053F14 product: protein tyrosine |
| | | phosphatase 4a3, full insert sequence |
| AK172192.1 | GI: 74182510 | Mus musculus activated spleen cDNA, RIKEN full- |
| | | length enriched library, clone: F830102P03 |
| | | product: protein tyrosine phosphatase 4a3, full insert |
| | | sequence |
| AK 143702.1 | GI: 74150753 | Mus musculus 6 days neonate spleen cDNA, |
| | | RIKEN full-length enriched library, clone: F43001 1 |
| | | C20 productprotein tyrosine phosphatase 4a3, full |
| | | insert sequence |
| AF035645.1 | GI: 2992631 | Mus musculus potentially prenylated protein |
| | | tyrosine phosphatase mPRL-3 (Prl3) mRNA, |
| | | complete cds |
| NM_008975.2 | GI: 31543526 | Mus musculus protein tyrosine phosphatase 4a3 |
| | | (Ptp4a3), mRNA |
| AK014601.1 | GI: 12852557 | Mus musculus 0 day neonate skin cDNA, RIKEN |
| | | full-length enriched library, clone: 4632430E19 |
| | | product:protein tyrosine phosphatase 4a3, full insert |
| | | sequence |
| AK004562.1 | GI: 12835815 | Mus musculus adult male lung cDNA, RIKEN full- |
| | | length enriched library, clone: 1200003F10 |
| | | productprotein tyrosine phosphatase 4a3, full insert |
| | | sequence |
| AK003954.1 | GI: 12834926 | Mus musculus 18-day embryo whole body cDNA, |
| | | RIKEN full-length enriched library, clone: |
| | | 1110029E17 product:protein tyrosine phosphatase |
| | | 4a3, full insert sequence |
| BC027445.1 | GI: 20071662 | Mus musculus protein tyrosine phosphatase 4a3, |
| | | mRNA (cDNA clone MGC: 36146 IMAGE: 4482106), |
| | | complete cds |

A “PRL-3 polypeptide” may comprise or consist of a human PRL-3 polypeptide, such as the sequence having Unigene accession number AF041434.1.

Homologues variants and derivatives thereof of any, some or all of these polypeptides are also included. For example, PRL-3 may include Unigene Accession Number BC066043.1.

PRL1

The following text is adapted from OMIM entry 604585.

PRL1 is also known as Protein-Tyrosine Phosphatase, Type 4a 1; PTP4A1, Phosphatase or Regenerating Liver 1, PTP(CAAX1). The chromosomal location of PRL1 is at gene map locus 6q12.

Cellular processes involving growth, differentiation, and metabolism are often regulated in part by protein phosphorylation and dephosphorylation. The protein tyrosine phosphatases (PTPs), which hydrolyze the phosphate monoesters of tyrosine residues, all share a common active site motif and are classified into three groups.

These include the receptor-like PTPs, the intracellular PTPs, and the dual-specificity PTPs, which can dephosphorylate at serine and threonine residues as well as at tyrosines.

Diamond et al 1994 Cell. Bio. 14:3752-3762, described a PTP from regenerating rat liver that is a member of a fourth class. The gene, which they designated PRL1, was one of many immediate early genes and expressed mainly in the nucleus. Over-expression of PRI1 in stably transfected cells resulted in a transformed phenotype, which suggested that it may play some role in tumorigenesis.

By using an in vitro prenylation screen, Cates et al., 1996, Cancer Lett. 110:49-55, isolated 2 human cDNAs encoding PRL1 homologs, designated PTP(CAAX1) and PTP(CAAX2) (PRL2; 601584), that are farnesylated in vitro by mammalian farnesyl:protein transferase. Overexpression of these PTPs in epithelial cells caused a transformed phenotype in cultured cells and tumor growth in nude mice. The authors concluded that PTP(CAAX1) and PTP(CAAX2) represent a novel class of isoprenylated, oncogenic PTPs.

Peng et al. 1998, J. Biol. Chem. 273: 17286-17295, reported that the human PTP(CAAX1) gene, or PRL1, is composed of 6 exons and contains 2 promoters. The predicted mouse, rat, and human PRL 1 proteins are identical. Zeng et al. 1998, Biochem. Biophys. Res. Commun. 244:421-427, determined that the human PRL1 and PRL2 proteins share 87% amino acid sequence identity. By FISH, Peng et al. (1998) mapped the PRL1 gene to 6q12.

Where the term “PRL-1” is used, this should be taken to refer to any PRL-1 sequence, including a PRL-1protein or a PRL-1nucleic acid and any fragment, variant homologue, derivative, variant thereof.

The properties and activities of PRL-1 are described in this document, for example, in the references.

Mouse and human PRL-1 proteins were described in detail in Zeng et al (1998), supra.

PRL-1 Sequences

The methods and compositions described here make use of PRL-1 polypeptides, which are described in detail below. As used here, the term “PRL-1” is intended to refer to a sequence set out in Table D1 below.

| Unigene | Description |
|---|---|
| NM 003463.3 | Homo sapiens protein tyrosine phosphatase type IV A, member 1 |
| | (PTP4A1), mRNA |
| CR602427.1 | full-length eDNA clone CSODK012YJ03 of HeLa cells Cot 25 normalized |
| | of Homo sapiens (human) |
| CR599216.1 | full-length cDNA clone CLOBB007ZF05 of Neuroblastoma of Homo |
| | sapiens (human) |
| CR596545.1 | full-length eDNA clone CSODK010YM06 of HeLa cells Cot 25-normalized |
| | of Homo sapiens (human) |
| CR749458.1 | Homo sapiens mRNA; eDNA DKFZp779M0721 (from clone |
| | DKFZp779M0721) |
| BC045571.1 | Homo sapiens protein tyrosine phosphatase type IV A, member 1, mRNA |
| | (eDNA clone MGC: 57320 IMAGE: 4826233), complete cds |
| AJ420505.1 | Homo sapiens mRNA full length insert eDNA clone EUROIMAGE 2096405 |
| AK312526.1 | Homo sapiens eDNA, FLJ92892 |
| BC023975.2 | Homo sapiens protein tyrosine phosphatase type IV A, member 1, mRNA |
| | (eDNA clone MGC: 1659 IMAGE: 2960001), complete cds |
| U69701.1 | Human protein tyrosine phosphatase hPRL-1N mRNA, partial cds |
| U48296.1 | Homo sapiens protein tyrosine phosphatase PTPCAAX 1 (hPTPCAAX1) |
| | mRNA, complete cds |
| AK081491.1 | Mus musculus 16 days embryo head eDNA, RIKEN full-length enriched |
| | library, clone: C 130021 BO 1 product:protein tyrosine phosphatase 4a1, |
| | full insert sequence |
| AK078120.1 | Mus musculus adult male medulla oblongata eDNA, RIKEN full-length |
| | enriched library, clone: 6330521 E 18 product: protein tyrosine |
| | phosphatase 4a1, full insert sequence |
| BC055039.1 | Mus musculus protein tyrosine phosphatase 4a1, mRNA (eDNA clone |
| | MGC: 62623 IMAGE: 6396041), complete cds |
| AK199907.1 | Mus musculus eDNA, clone: Y1G0132L24, strand:minus, |
| | reference: ENSEMBL:Mouse-TranscriptENST: ENSMUST00000061959, |
| | based on BLAT search Mus musculus eDNA, clone: Y1G0129D05, |
| | strand: plus, reference: ENSEMBL:Mouse-Transcript- |
| AK198788.1 | ENST: ENSMUST00000061959, based on BLAT search Mus musculus |
| | eDNA, clone: Y1G0109N22, strand: plus, reference: ENSEMBL:Mouse- |
| | Transcript- |
| AK192767.1 | ENST: ENSMUST00000055216, based on BLAT search Mus musculus |
| | eDNA, clone: YOG0140011, strand: plus, reference: ENSEMBL:Mouse- |
| | Transcript- |
| AK187266.1 | ENST: ENSMUST00000061959, based on BLAT search Mus musculus |
| | protein tyrosine phosphatase 4a1, mRNA (eDNA clone BC086787.1 |
| | MGC:102117 IMAGE: 30538771) |
| BC094447.1 | complete cds Mus musculus protein tyrosine phosphatase 4a1, mRNA |
| | (eDNA clone MGC: 102501 IMAGE: 3990529), complete cds |
| AK 150506.1 | Mus musculus bone marrow macrophage eDNA, RIKEN full-length |
| | enriched library, clone: I830008L20 product: protein tyrosine phosphatase |
| | 4a 1, full insert sequence |
| AK 148288.1 | full Mus musculus B16 F10Y cells eDNA, RIKEN full-length enriched |
| | library, clone: G3 70079M23 product: protein tyrosine phosphatase 4a1, |
| | insert sequence |
| AK151533.1 | Mus musculus bone marrow macrophage cDNA, RIKEN full-length |
| | enriched library, clone: I830031H07 product:protein tyrosine phosphatase |
| | 4a1, full insert sequence |
| U84411.1 | Mus musculus protein tyrosine phosphatase (PRL-1) mRNA, complete |
| | cds |
| NM 011200.2 | Mus musculus protein tyrosine phosphatase 4a1 (Ptp4a1), mRNA |
| | Mus musculus, protein tyrosine phosphatase 4a1, clone |
| BC031734.1 | IMAGE: 3590144, mRNA Mus musculus, protein tyrosine phosphatase 4a |
| | 1, clone |
| BC003761.1 | IMAGE: 3157812, mRNA |

A “PRL-1 polypeptide” may comprise or consist of a human PRL-1 polypeptide, such as the sequence having Unigene accession number NM_003463.3. Homologues variants and derivatives thereof of any, some or all of these polypeptides are also included. For example, PRL-1 may include Unigene Accession Number U84411.1.

VHZ

The methods and compositions described here make use of VHZ, which is described in detail below.

VHZ is also known as DUSP23, MOSP, LDP-3, DUSP25, FLJ20442 and RP1 1-190A12.1

As used here, the term “VHZ” may refer to a polypeptide sequence having GenBank Accession number NP_060293.2, NP_081001.1, XP_341 157.1, XP_001 170819.1, XP_001 170835.1, XP_545747.2, NP_001076078.1, NP 00101 1371.1, NP_783859.1, NP_001034709.1, XP_001480730.1, XP_001 1 17253.1 or XP_001 1 17256.1.

A “VHZ polypeptide” may comprise or consist of a human VHZ polypeptide, such as the sequence having accession number NP 060293.

With regard to nucleic acid sequences, the terms “VHZ polynucleotide”, “VHZ nucleotide” and “VHZ nucleic acid” may be used interchangeably, and should be understood to specifically include both cDNA and genomic VHZ sequences. These terms are also intended to include a nucleic acid sequence capable of encoding a VHZ polypeptide and/or a fragment, derivative, homologue or variant of this.

Where reference is made to a VHZ nucleic acid, this should be taken as a reference to any member of the VHZ family of nucleic acids. Of particular interest are VHZ nucleic acids selected from the group consisting of: NM_017823.3, NM_026725.2, XM_341 156.3, XM_001 170819.1, XM_170835.1, XM_545747.2, NM_001082609.1, NM_00101 1371.1, N _175732.1, NM_001039620.1, XM_001480680.1, XM_001 1 17253.1 or XM 001 1 17256.1.

Also included are any one or more of the nucleic acid sequences set out as “Other VHZ nucleic acid sequences” below.

For example, the VHZ nucleic acid may comprise a human VHZ sequence having GenBank Accession Number NM 017823.3.

Her2

Her2/neu (also known as ErbB-2) stands for “human epidermal growth factor receptor 2” and is a protein giving higher aggressiveness in breast cancers. It is a member of the ErbB protein family, more commonly known as the epidermal growth factor receptor family. HER2/neu has also been designated as CD340 (cluster of differentiation 340) and p185. HER2 is a cell membrane surface-bound receptor tyrosine kinase and is normally involved in the signal transduction pathways leading to cell growth and differentiation.

As described herein, HER2 may refer to a polypeptide sequence selected from GenBank accession numbers NP_004439.2, NP_001005862.1, NP_001003817.1, AAI67147.1

A “Her2 polypeptide” as referred to herein may comprise or consist of a human HER2 polypeptide sequence, such as that of accession number P04626.1

Her2 polypeptides are described in U.S. Pat. No. 6,333,169 and EP1418235.

Other oncoproteins useful in the invention include EGFR (GenBank accession numbers CAA25240 (GI:119533), ADZ75461.1 (GI326467049)), SHP1 (GenBank accession numbers NP002822.2 (GI: 18104989), NP536858.1 (GI: 18104991), NP536859.1 (GI: 18104991)), Tiam (GenBank accession numbers NP003244.2 (GI: 115583670), AAA98443.1 (GI: 897557), Q13009.2 (GI: 152031709)), Myc (GenBank accession numbers AAA59886.1 (GI: 188975), AAA59887.1 (GI: 188977), CAA25015.2 (GI: 29839758), NP002458.2 (GI: 71774083)), Ras (GenBank accession number AAA34557.1 (GI: 171374)) and Runx-1 (GenBank accession number NP001079966.1 (GI: 148232064)).

Estrogen Receptor

An oncoprotein useful in the present invention is Estrogen Receptor (ER). The oncoprotein may be human ER. It may comprise or consist of the protein sequence set out at P03372 (GI: 544257).

Estrogen Receptor and fragments thereof will be useful for treatment of breast cancer caused by, or associated with, overexpression of estrogen receptor (ER). Antibodies against ER or vaccination using ER oncoprotein or a fragment thereof could be used to prevent spreading. This is particularly useful to target ER positive breast cancer patients regardless of the expression of Her2 or other proteins.

Estrogen Receptor (ER) is a ligand-activated transcription factor composed of several domains important for hormone binding, DNA binding, and activation of transcription. Alternative splicing results in several ER mRNA transcripts, which differ primarily in their 5-prime untranslated regions. The translated receptors show less variability (see OMIM reference 133430).

Hepatitis B Virus (HBV) Proteins

Hepatitis B proteins may be suitable for use in the invention. HBV exists as 8 genotypes.

For example, the HBV X-protein. HBV X-protein is localized in the nucleus of infected cells. Most hepatocellular carcinoma (HCC) are associated with HBV infection. Accordingly, HBV proteins may be useful for treating HCC, antibody targeting viral protein to specifically destroy virally infected cells whilst leaving normal cells unharmed.

Protein sequences for HBV-X protein have been deposited at GenBank and are suitable for use in the present invention. For example, the term “HBV-X protein” may be used to refer to a protein comprising or consisting of the sequence set out at GenBank CBX46805.1 (GI: 310923520), or EMBL accession FR714506.1, or a protein encoded by a gene having a sequence as set out at Accession AB670311.1 (GI: 371919030).

Other oncoproteins that are particularly preferred herein are c-myc (CAA46984.1 GI:396512), H-ras (CAG38816.1 GI:49168642), AKT-1 (AAL55732.1 GI:18027298), p53 (BAC16799.1 GI:23491729 or a variant thereof, such as NP_000537.3 GI:120407068), Rac1 (CAB53579.5 GI:8574038), FAK (NP_722560.1 GI:24476013 or NP_005598.3 GI:27886593), Runx1 (AAI36381.1 GI: 223459612), PTEN (AAD13528.1 GI: 4240387), b-actin (NP_001092.1 GI:4501885) or GAPDH (NP_001243728.1 GI:378404908), N-Cadherin (CAA40773.1 GI:1335229), PDGF receptor (alpha) (NP_006197.1 GI:5453870), FLT-3 (NP_004110.2 GI:121114304) or EGFR or p-EGFR (NP_005219.2 GI:29725609).

Polypeptide

A “polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids.

“Polypeptides” include amino acid sequences modified either by natural processes, such as post-translational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications.

Polypeptides may be branched as a result of ubiquitination, and they may be cyclic, with or without branching. Cyclic, branched and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, Proteins—Structure and Molecular Properties, 2nd Ed., T. E. Creighton, W. H. Freeman and Company, New York, 1993 and Wold, F., Posttranslational Protein Modifications: Perspectives and Prospects, pgs. 1-12 in Posttranslational Covalent Modification of Proteins, B. C. Johnson, Ed., Academic Press, New York, 1983; Seifter et al., “Analysis for protein modifications and nonprotein cofactors”, Meth Enzymol (1990) 182:626-646 and Rattan et al, “Protein Synthesis: Posttranslational Modifications and Aging”, Ann NY Acad Sci (1992) 663:48-62.

The term “polypeptide” includes the various synthetic peptide variations known in the art, such as a retroinverso D peptides. The peptide may be an antigenic determinant and/or a T-cell epitope. The peptide may be immunogenic in vivo. The peptide may be capable of inducing neutralising antibodies in vivo.

As applied to intracellular oncoproteins, the resultant amino acid sequence may have one or more activities, such as biological activities in common with a intracellular oncoprotein polypeptide, for example a human intracellular oncoprotein. For example, a intracellular oncoprotein homologue may have an increased expression level in cancer cells compared to normal breast cells. In particular, the term “homologue” covers identity with respect to structure and/or function providing the resultant amino acid sequence has intracellular oncoprotein activity. With respect to sequence identity (i.e. similarity), there may be at least 70%, such as at least 75%, such as at least 85%, such as at least 90% sequence identity. There may be at least 95%, such as at least 98%, sequence identity. These terms also encompass polypeptides derived from amino acids which are allelic variations of the intracellular oncoprotein nucleic acid sequence.

Where reference is made to the “activity” or “biological activity” of a polypeptide such as an intracellular oncoprotein, these terms are intended to refer to the metabolic or physiological function of the intracellular oncoprotein, including similar activities or improved activities or these activities with decreased undesirable side effects. Also included are antigenic and immunogenic activities of the intracellular oncoprotein. Examples of such activities, and methods of assaying and quantifying these activities, are known in the art, and are described in detail elsewhere in this document.

Variants, Derivatives and Homologues

The methods described herein may involve the detection or quantification of PRL3 polypeptides, or variants, homologues or derivatives of such peptides. The methods described herein may involve the detection of an oncoprotein that is not identical to the sequence disclosed herein, but may carry one or more mutations relative to a known sequence. Thus, such sequences are not limited to the particular sequences set forth in this document, but also include homologous sequences, for example related cellular homologues, homologues from other species and variants or derivatives thereof.

The terms “variant”, “homologue” or “derivative” in relation to a nucleotide sequence described in this document include any substitution of, variation of, modification of, replacement of, deletion of or addition of one (or more) nucleotides from or to the sequence. The resulting sequence may be capable of encoding a polypeptide which has intracellular oncoprotein binding activity as described elsewhere in this document.

As indicated above, with respect to sequence identity, a “homologue” has such as at least 5% identity, at least 10% identity, at least 15% identity, at least 20% identity, at least 25% identity, at least 30% identity, at least 35% identity, at least 40% identity, at least 45% identity, at least 50% identity, at least 55% identity, at least 60% identity, at least 65% identity, at least 70% identity, at least 75% identity, at least 80% identity, at least 85% identity, at least 90% identity, or at least 95% identity to a relevant sequence.

There may be at least 95% identity, such as at least 96% identity, such as at least 97% identity, such as at least 98% identity, such as at least 99% identity. Nucleotide homology comparisons may be conducted as described above. A sequence comparison program such as the GCG Wisconsin Bestfit program described above may be used for this purpose. The default scoring matrix has a match value of 10 for each identical nucleotide and −9 for each mismatch. The default gap creation penalty is −50 and the default gap extension penalty is −3 for each nucleotide.

Patient

The patient to be treated may be any animal or human. The patient is preferably a non-human mammal, more preferably a human patient. The patient may be male or female. The patient may have, or may be suspected of having a cancer. In some cases the patient has not been previously diagnosed as having cancer. In some cases, the patient does not exhibit symptoms associated with cancer. In some cases, the patient is known to have a predisposition to cancer, such as a family history of cancer, or lifestyle indicators of cancer. In some cases the individual or patient has been previously diagnosed as having cancer.

The terms patient and subject are used interchangably herein.

Cancer

The methods disclosed herein relate to the diagnosis, prognosis, treatment or prevention of cancer. The cancer may be a PRL3 expressing cancer. The cancer may be a PRL3 overexpressing cancer (i.e. a cancer that expresses PRL3 at an elevated level as compared to a non-cancerous tissue, or at an elevated level as compared to other cancerous tissue).

In particular, the methods disclosed herein relate to gastric cancer or stomach cancer. The methods may relate to nasopharyngeal cancer, bladder cancer, lung cancer, breast cancer or prostate cancer. Some methods relate to colon cancer or uveal melanoma. The cancer may be a primary cancer or a metastatic cancer.

Also as referred to herein, a “cancer” can comprise any one or more of the following: acute lymphocytic leukemia (ALL), acute myeloid leukemia (AML), adrenocortical cancer, anal cancer, bladder cancer, blood cancer, bone cancer, brain tumor, breast cancer, cancer of the female genital system, cancer of the male genital system, central nervous system lymphoma, cervical cancer, childhood rhabdomyosarcoma, childhood sarcoma, chronic lymphocytic leukemia (CLL), chronic myeloid leukemia (CML), colon and rectal cancer, colon cancer, endometrial cancer, endometrial sarcoma, esophageal cancer, eye cancer, gallbladder cancer, gastric cancer, gastrointestinal tract cancer, hairy cell leukemia, head and neck cancer, hepatocellular cancer, Hodgkin's disease, hypopharyngeal cancer, Kaposi's sarcoma, kidney cancer, laryngeal cancer, leukemia, liver cancer, lung cancer, malignant fibrous histiocytoma, malignant thymoma, melanoma, mesothelioma, multiple myeloma, myeloma, nasal cavity and paranasal sinus cancer, nasopharyngeal cancer, nervous system cancer, neuroblastoma, non-Hodgkin's lymphoma, oral cavity cancer, oropharyngeal cancer, osteosarcoma, ovarian cancer, pancreatic cancer, parathyroid cancer, penile cancer, pharyngeal cancer, pituitary tumor, plasma cell neoplasm, primary CNS lymphoma, prostate cancer, rectal cancer, respiratory system, retinoblastoma, salivary gland cancer, skin cancer, small intestine cancer, soft tissue sarcoma, stomach cancer, testicular cancer, thyroid cancer, urinary system cancer, uterine sarcoma, vaginal cancer, vascular system, Waldenstrom's macroglobulinemia and Wilms' tumor.

Cancers may be of a particular type. Examples of types of cancer include astrocytoma, carcinoma (e.g. adenocarcinoma, hepatocellular carcinoma, medullary carcinoma, papillary carcinoma, squamous cell carcinoma), glioma, lymphoma, medulloblastoma, melanoma, myeloma, meningioma, neuroblastoma, sarcoma (e.g. angiosarcoma, chrondrosarcoma, osteosarcoma).

Sample

Methods described herein may be performed on a sample that has been obtained from a patient. Such methods may thus be performed ex vivo. They may be performed in vitro.

Preferably, samples useful in the methods described herein are urine samples.

In some arrangements the sample is taken from a bodily fluid, more preferably one that circulates through the body. Accordingly, the sample may be a blood sample or lymph sample. Other bodily fluids suitable in methods of the invention include serum, urine, saliva and breast milk.

The sample may comprise or may be derived from: a quantity of blood; a quantity of serum derived from the individual's blood which may comprise the fluid portion of the blood obtained after removal of the fibrin clot and blood cells; a quantity of plasma; a quantity of pancreatic juice; a tissue sample or biopsy; or cells isolated from said individual.

The sample may be a blood sample or blood-derived sample. The blood derived sample may be a selected fraction of a patient's blood, e.g. a selected cell-containing fraction or a plasma or serum fraction.

The sample may be a sample of exosomes prepared from a sample of bodily fluid, such as a sample of exosomes from urine, plasma or saliva. The sample may be a sample in which the proportion of exosomes is increased (i.e. the exosomes have been concentrated) relative to the sample that was extracted from the patient. In some cases, the sample contains substantially only exosomes.

Diagnosis

Diagnosis refers to the identification of a disease, such as cancer. Methods described herein may be used to detect a cancer. They may be used to diagnose a subtype or subclass, or stage, of a particular cancer.

Detection of oncoproteins in a sample in accordance with the methods of the present invention may be used for the purpose of diagnosis of a cancerous condition in the patient, diagnosis of a predisposition to a cancerous condition or for determining a prognosis (prognosticating) of a cancerous condition. The diagnosis or prognosis may relate to an existing (previously diagnosed) cancerous condition, which may be benign or malignant, or may relate to a suspected cancerous condition or may relate to the screening for cancerous conditions in the patient (which may be previously undiagnosed).

Other diagnostic tests may be used in conjunction with those described here to enhance the accuracy of diagnosis or prognosis of a cancerous condition or to confirm a result obtained by using the tests described here.

The method of diagnosis may be an in vitro method performed on the patient sample, or following processing of the patient sample. Once the sample is collected, the patient is not required to be present for the in vitro method of diagnosis to be performed and therefore the method may be one which is not practised on the human or animal body.

Other diagnostic tests may be used in conjunction with those described here to enhance the accuracy of the diagnosis or prognosis or to confirm a result obtained by using the tests described here.

Prognosis

Prognosis, prognosing and prognose refer to estimating the risk of future outcomes in an individual based on their clinical and non-clinical characteristics. In particular, a method of determining the prognosis as used herein refers to the prediction of the outcome of, or future course of, an individual's or patient's cancer. Prognosis includes the prediction of patient's survival. Prognosis may be useful for determining an appropriate therapeutic treatment. Prognostic testing may be undertaken with (e.g. at the same time as) the diagnosis of a previously undiagnosed cancerous condition, or may relate to an existing (previously diagnosed) condition.

The method of prognosis may be an in vitro method performed on the patient sample, or following processing of the patient sample. Once the sample is collected, the patient is not required to be present for the in vitro method of prognosis to be performed and therefore the method may be one which is not practised on the human or animal body.

As disclosed herein, the level of oncoprotein in the sample may be used to indicate the prognosis of patient's cancer. As described herein, elevated oncoprotein expression and activity may correlate with a later stage or more advanced cancer, and may indicate a shorter overall survival. Thus, increased level of oncoprotein may indicate poor prognosis such as reduced survival time.

Prognosis may be used to predict the disease free survival time of an individual, progression-free survival time, disease specific survival time, survival rate, or survival time.

Patient Selection

Methods disclosed herein include the selection or classification of patients suitable of treatment. For example, the methods may be useful for selecting or classifying patients suitable for treatment with anti-PRL3 antibody therapy. As used herein, subjects who are considered suitable for treatment are those subjects who are expected to benefit from, or respond to, the treatment. Subjects may have, or be suspected of having, or be at risk of having cancer. Subjects may have received a diagnosis of cancer. In particular, subjects may have, or be suspected of having, or be at risk of having, cancer. In some aspects, patients are selected on the basis of the presence of an oncoprotein in a sample, or the amount of oncoprotein expression, such as the presence or amount of PRL3 in a sample of urine from the patient. Also disclosed are methods of treating patients selected by these methods.

In some cases, the observation that the biomarker protein, such as PRL3, is overexpressed in the sample as compared to a sample from a non-cancerous patient may be indicative that the patient is suitable for treatment. In other cases, the presence of the biomarker protein, as compared to the absence in a sample from a non-cancerous patient may be indicative that the patient is suitable for treatment.

Detection and Quantification

Methods disclosed herein involve the detection and/or quantification of oncoproteins. Detection, as used herein, refers to measurement of oncoprotein without quantification. Methods for detection and quantification of PRL3 nucleotides and proteins are well known in the art and will be readily appreciated by a skilled person.

Protein, for example, may be detected or quantified by immunoassay. Immunoassay methods are well known in the art and will generally comprise: (a) providing a polypeptide comprising an epitope bindable by an antibody against said protein; (b) incubating a biological sample with said polypeptide under conditions which allow for the formation of an antibody-antigen complex; and (c) determining whether antibody-antigen complex comprising said polypeptide is formed. Immunoassay methods include western blotting and ELISA.

Immunoassays include, but are not limited to, Enzyme-linked immunosorbent assay (ELISA), lateral flow test, latex agglutination, other forms of immunochromatography, western blot, and/or magnetic immunoassay.

Protein may also be detected or quantified using mass spectrometry. For example, mass spectrometry using electrospray ionization (ESI) or matrix-assisted laser desorption/ionisation (MALDI).

Other methods of protein quantification include spectroscopy based methods. Such methods may involve colorimetric assays or spectrophotometric assays.

Methods for detecting and quantifying nucleic acids are well known in the art. Methods include polymerase chain reaction (PCR) based methods and hybridization methods.

Polymerase chain reaction based methods include PCR, reverse transcription PCR (RT-PCR and quantitative RT-PCR. Such methods utilise a primer, or short DNA fragment which binds specifically to a DNA sequence of interest. RNA may be transcribed to DNA before or during the method.

Elevated Expression or Activity

As disclosed herein, elevated oncoprotein expression may be indicative of a poor prognosis for cancer patients. As used herein, elevated expression is used interchangeably with increased expression, high expression or high level. Elevated expression of a protein may correlate with elevated level of that protein in urine.

Elevated expression means an increase in the level of oncoprotein. The expression may be elevated locally or globally, for example within a particular tissue or cell type, such as within a tumor or within bone marrow, or maybe elevated throughout the body of the patient. Elevated expression may be caused by an increase in production of that protein or nucleic acid, or by a decrease in the elimination or destruction of that protein or nucleic acid, or both.

Elevated activity may be caused by an increase in the amount of the protein or nucleic acid, or by an increase in the activity of each individual molecule. This may occur through a mutation in the gene or protein sequence, such as an activating mutation, or may be due to a post-translational change, such as aberrant protein phosphorylation.

In some cases, the expression of oncoprotein is significantly upregulated in the patient or sample, relative to the expression in a non-cancerous individual or a non-cancerous tissue.

Overexpression or increased activity of oncoprotein relative to a control may be indicative of a poor prognosis and poor survival. Very high overexpression or very high activity of oncoprotein may be indicative of a very poor prognosis, and very poor survival.

In some cases, expression or activity of 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, 750% or 1000% or a higher percentage more than the expression or activity in the control is indicative of a poor prognosis.

In some cases, expression or activity of 1.5 times, 2 times, 3 times, 4 times, 5 times, 6 times, 7 times, 8 times, 9 times, 10 times, 15 times, 20 times, 25 times, 30 times, 35 times, 40 times, 45 times, 50 times, 100 times, or more times more than the expression or activity in the control is indicative of a poor prognosis.

Control

In some cases, the method involves comparing oncoprotein in a sample from a patient with oncoprotein in one or more control samples.

The comparison may not require the analysis of the control sample to be simultaneously or sequentially performed with the analysis of the sample from the patient. Instead, the comparison may be made with results previously obtained from a control sample, such as results stored in a database.

The control sample may be a sample obtained from the patient prior to the onset of cancer, or prior to the observation of symptoms associated with cancer.

The control sample may be a sample obtained from another individual, such as an individual who does not have cancer. The individual may be matched to the patient according to one or more characteristics, for example, sex, age, medical history, ethnicity, weight or expression of a particular marker. The control sample may have been obtained from the bodily location, or be of the same tissue or sample type as the sample obtained from the patient.

The control sample may be a collection of samples, thereby providing a representative value across a number of different individuals or tissues.

In some cases, the control may be a reference sample or reference dataset. The reference may be a sample that has been previously obtained from a subject with a known degree of suitability for a particular treatment. The reference may be a dataset obtained from analyzing a reference sample.

Controls may be positive controls in which the target molecule is known to be present, or expressed at high level, or negative controls in which the target molecule is known to be absent or expressed at low level.

Controls may be samples of tissue that are from subjects who are known to benefit from the treatment. The tissue may be of the same type as the sample being tested. For example, a sample of tumor tissue from a subject may be compared to a control sample of tumor tissue from a subject who is known to be suitable for the treatment, such as a subject who has previously responded to the treatment.

In some cases the control may be a sample obtained from the same subject as the test sample, but from a time when the subject known to be healthy, such as a time when the subject was known to be free from cancer. Thus, a sample of cancerous tissue from a subject may be compared to a non-cancerous tissue sample.

In some cases, the control is a cell culture sample.

In some cases, a test sample is analysed prior to incubation with an antibody to determine the level of background staining inherent to that sample.

In some cases an isotype control is used. Isotype controls use an antibody of the same class as the target specific antibody, but are not immunoreactive with the sample. Such controls are useful for distinguishing non-specific interactions of the target specific antibody.

Anti-Oncoprotein Antibodies

Antibodies useful in the methods disclosed herein can be made by any methods known in the art. The term antibody as used herein includes whole antibodies and antibody fragments, humanised, chimeric, and recombinant antibodies. The antibodies may be monoclonal or polyclonal.

Antibodies which will bind to PRL1 and PRL3 are known. For example, as disclosed in Li et al., 2005.

The antigen-binding portion may be a part of an antibody (for example a Fab fragment) or a synthetic antibody fragment (for example a single chain Fv fragment [ScFv]). Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimeric antibodies are discussed by Neuberger et al (1988, 8th International Biotechnology Symposium Part 2, 792-799).

Monoclonal antibodies (mAbs) are useful in the methods of the invention and are a homogenous population of antibodies specifically targeting a single epitope on an antigen. Suitable monoclonal antibodies can be prepared using methods well known in the art (e.g. see Köhler, G.; Milstein, C. (1975). “Continuous cultures of fused cells secreting antibody of predefined specificity”. Nature 256 (5517): 495; Siegel D L (2002). “Recombinant monoclonal antibody technology”. Schmitz U, Versmold A, Kaufmann P, Frank H G (2000); “Phage display: a molecular tool for the generation of antibodies—a review”. Placenta. 21 Suppl A: S106-12. Helen E. Chadd and Steven M. Chamow; “Therapeutic antibody expression technology,” Current Opinion in Biotechnology 12, no. 2 (Apr. 1, 2001): 188-194; McCafferty, J.; Griffiths, A.; Winter, G.; Chiswell, D. (1990). “Phage antibodies: filamentous phage displaying antibody variable domains”. Nature 348 (6301): 552-554; “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimeric antibodies are discussed by Neuberger et al (1988, 8th International Biotechnology Symposium Part 2, 792-799)).

Polyclonal antibodies are useful in the methods of the invention. Monospecific polyclonal antibodies are preferred. Suitable polyclonal antibodies can be prepared using methods well known in the art.

Fragments of antibodies, such as Fab and Fab2 fragments may also be used as can genetically engineered antibodies and antibody fragments. The variable heavy (VH) and variable light (VL) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sd. USA 81, 6851-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the VH and VL partner domains are linked via a flexible oligopeptide (Bird et al (1988) Science 242, 423; Huston et al (1988) Proc. Natl. Acad. Sd. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

By “ScFv molecules” we mean molecules wherein the VH and VL partner domains are covalently linked, e.g. directly, by a peptide or by a flexible oligopeptide. Fab, Fv, ScFv and dAb antibody fragments can all be expressed in and secreted from E. coli, thus allowing the facile production of large amounts of the said fragments.

Whole antibodies, and F(ab′)2 fragments are “bivalent”. By “bivalent” we mean that the said antibodies and F(ab′)2 fragments have two antigen combining sites. In contrast, Fab, Fv, ScFv and dAb fragments are monovalent, having only one antigen combining site. Synthetic antibodies which bind to PRL3 may also be made using phage display technology as is well known in the art (e.g. see “Phage display: a molecular tool for the generation of antibodies—a review”. Placenta. 21 Suppl A: S106-12. Helen E. Chadd and Steven M. Chamow; “Phage antibodies: filamentous phage displaying antibody variable domains”. Nature 348 (6301): 552-554).

In some preferred embodiments the antibody is detectably labelled or, at least, capable of detection. For example, the antibody may be labelled with a radioactive atom or a coloured molecule or a fluorescent molecule or a molecule which can be readily detected in any other way. Suitable detectable molecules include fluorescent proteins, luciferase, enzyme substrates, and radiolabels. The antibody may be directly labelled with a detectable label or it may be indirectly labelled. For example, the antibody may be unlabelled and can be detected by another antibody which is itself labelled. Alternatively, the second antibody may have bound to it biotin and binding of labelled streptavidin to the biotin is used to indirectly label the first antibody.

The antibody may be an antibody capable of binding epitope KAKFYN and/or HTHKTR. The antibody may be an antibody having a sequence identical to mouse anti-PRL3 antibody from hybridoma clone 223 or hybridoma clone 318, as reported by Li et al 2005. The antibody may compete for target binding with the antibody from hybridoma clone 223 or hybridoma clone 318 described in Li et al 2005. The antibody may be a humanised antibody, a chimeric antibody or a fully human antibody. The antibody be homologous to an antibody described herein.

As indicated above, with respect to sequence identity, a “homologue” has such as at least 5% identity, at least 10% identity, at least 15% identity, at least 20% identity, at least 25% identity, at least 30% identity, at least 35% identity, at least 40% identity, at least 45% identity, at least 50% identity, at least 55% identity, at least 60% identity, at least 65% identity, at least 70% identity, at least 75% identity, at least 80% identity, at least 82% identity, at least 84% identity, at least 86% identity, at least 88% identity, at least 90% identity, at least 92% identity, at least 94% identity, at least 96% identity, or at least 98% identity to a relevant sequence. The relevant sequence may be the CDR sequence, or across the sequence of the heavy and/or light variable chain.

Methods

Methods according to the present invention may be performed in vitro or ex vivo. The term “in vitro” is intended to encompass experiments with materials, biological substances, cells and/or tissues in laboratory conditions or in culture. “Ex vivo” refers to something present or taking place outside an organism, e.g. outside the human or animal body, which may be on tissue (e.g. whole organs) or cells taken from the organism.

The methods disclosed herein relate to the determination of protein expression. Protein expression can be measured by quantifying the amount of protein in a cell, tissue or sample, or by observing the localisation of the protein within cells and tissues.

In some cases, immunoassays are used to detect the target (e.g. PRL3) in a sample from the subject. Immunoassays use antibodies with specific affinity for the target molecule in conjunction with a detectable molecule. In some cases, the antibody is conjugated to the detectable molecule. The detectable molecule may be referred to as a label. The detectable molecule produces a detectable signal when the antibody is bound to the target molecule. The detectable signal may be a quantifiable signal. In some cases, an aptamer is used instead of, or together with, the antibody. Immunoassays include immunohistochemistry, ELISA, immunoblotting and flow cytometry. In certain aspects described herein, the assay is an immunohistochemistry assay. Such assays commonly use antibodies, although other target specific molecules such as aptamers or other ligands may be used.

The method may be approved for use by a regulatory agency. The method may be an FDA approved method.

ELISA

In some cases, the target may be detected by ELISA (enzyme-linked immunosorbent assay). Target molecules from a sample are attached to a surface and detected using a specific antibody. The target may be attached to the surface non-specifically (via adsorption to the surface) or specifically (using a specific capture agent such as an antibody). ELISA may be used to quantify target in a sample. ELISA is particularly suited to the analysis of liquid samples, such as serum, urine or saliva.

Immunoblotting

In some aspects, the target is detected by immunoblotting, or western blotting. In such methods, proteins in a sample are separated based on their electrical charge or size. They may be separated by an electrophoresis based method. The separated proteins are transferred to a membrane, where they are stained with an antibody that is specific to the target. The antibody is then detected, either directly by virtue of the antibody being conjugated to a detectable label, or indirectly, by adding a labelled secondary antibody.

Treatment

The methods of diagnosis disclosed herein may be used to guide choices of therapy. The methods may be used to select a patient for treatment, such as for a particular type of treatment. The methods may be used to monitor the progress, or success, of a particular therapy. The methods may be used to select a patient for treatment. Also disclosed herein are methods of treatment, the methods involving treating a patient selected for treatment based on the presence or level of one or more oncoproteins in a urine sample from the patient.

The treatment may result in an alleviation of the symptoms of the cancer, or may result in the complete treatment of the cancer. The treatment may slow the progression of the cancer, or may prevent the worsening of the symptoms of the cancer. In particularly preferred embodiments, the methods may be used to select patients for treatment with, or monitor the progress or success of treatment with, anti-intracellular oncoprotein antibody, such as an anti-PRL3 antibody.

Medicaments and pharmaceutical compositions according to aspects of the present invention may be formulated for administration by a number of routes, including but not limited to, parenteral, intravenous, intra-arterial, intramuscular, intratumoural, oral and nasal. The medicaments and compositions may be formulated in fluid or solid form. Fluid formulations may be formulated for administration by injection to a selected region of the human or animal body.

Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

A treatment may involve administration of more than one therapeutic agent. An agent may be administered alone or in combination with other treatments, either simultaneously or sequentially dependent upon the condition to be treated. For example, the treatment may be a co-therapy involving administration of two agents, one or more of which may be intended to treat the cancer. Thus, anti-PRL3 antibody may be administered with another drug, such as a chemotherapeutic agent, prodrug, antibody or hormone treatment. The treatment may additionally involve radiotherapy.

Examples of treatments and therapies include, but are not limited to, chemotherapy (the administration of active agents, including, e.g. drugs, such as chemotherapeutics); surgery; and radiation therapy.

A “chemotherapeutic agent” is a chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinase inhibitors. Chemotherapeutic agents include compounds used in “targeted therapy” and conventional chemotherapy.

Examples of chemotherapeutic agents include: Lenalidomide (REVLIMID®, Celgene), Vorinostat (ZOLINZA®, Merck), Panobinostat (FARYDAK®, Novartis), Mocetinostat (MGCD0103), Everolimus (ZORTRESS®, CERTICAN®, Novartis), Bendamustine (TREAKISYM®, RIBOMUSTIN®, LEVACT®, TREANDA®, Mundipharma International), erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo [4.3.0] nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethylethanamine, NOLVADEX®, ISTUBAL®, VALODEX®), and doxorubicin (ADRIAMYCIN®), Akti-1/2, HPPD, and rapamycin.

More examples of chemotherapeutic agents include: oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, Ill.), vandetanib (rINN, ZD6474, ZACTIMA®, AstraZeneca), chloranmbucil, AG1478, AG1571 (SU 5271; Sugen), temsirolimus (TORISELφ, Wyeth), pazopanib (GlaxoSmithKline), canfosfamide (TELCYTA®, Telik), thiotepa and cyclosphosphamide (CYTOXAN®, NEOSAR®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g. calicheamicin, calicheamicin gamma1I, calicheamicin omegall (Angew Chem. Intl. Ed. Engl. (1994) 33:183-186); dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, nemorubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; 6-thioguanine; mercaptopurine; methotrexate; platinum analogs such as cisplatin and carboplatin; vinblastine; etoposide (VP-16); ifosfamide; mitoxantrone; vincristine; vinorelbine (NAVELBINE®); novantrone; teniposide; edatrexate; daunomycin; aminopterin; capecitabine (XELODA®, Roche); ibandronate; CPT-11; topoisomerase inhibitor RFS 2000; difluoromethylornithine (DMFO); retinoids such as retinoic acid; and pharmaceutically acceptable salts, acids and derivatives of any of the above. Combinations of agents may be used, such as CHP (doxorubicin, prednisone, cyclophosphamide), or CHOP (doxorubicin, prednisone, cyclophopsphamide, vincristine).

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors such as MEK inhibitors (WO 2007/044515); (v) lipid kinase inhibitors; (vi) antisense oligonucleotides, particularly those which inhibit expression of genes in signaling pathways implicated in aberrant cell proliferation, for example, PKC-alpha, Raf and H-Ras, such as oblimersen (GENASENSE®, Genta Inc.); (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; topoisomerase 1 inhibitors such as LURTOTECAN®; ABARELIX® rmRH; (ix) anti-angiogenic agents such as bevacizumab (AVASTIN®, Genentech); and pharmaceutically acceptable salts, acids and derivatives of any of the above.

Also included in the definition of “chemotherapeutic agent” are therapeutic antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), ofatumumab (ARZERRA®, GSK), pertuzumab (PERJETA™, OMNITARG™, 2C4, Genentech), trastuzumab (HERCEPTIN®, Genentech), tositumomab (Bexxar, Corixia), MDX-060 (Medarex) and the antibody drug conjugate, gemtuzumab ozogamicin (MYLOTARG®, Wyeth).

Humanized monoclonal antibodies with therapeutic potential as chemotherapeutic agents in combination with the conjugates of the invention include: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

Treatment Monitoring

Also disclosed herein are methods of monitoring the success of a therapeutic treatment. Such methods involve the comparison of two or more samples from the same patient at different time points. For example, a sample may be taken prior to, or at the time of, commencing treatment. Such sample may be compared to a sample taken after commencement of treatment, such as after 1 week, 1 month, 2 months, 3 months, 4 months, 5 months, 6 months, 7 months, 8 months, 9 months, 10 months, 11 months, 12 months, 14 months, 16 months, 18 months or 2 years after commencing treatment. A decrease in the level of biomarker in the urine may be indicative that the treatment is successful. An increase in the level of biomarker in urine, or no significant change in the level of biomarker may indicate that a more intensive treatment is required, such as an increased dosage of therapeutic agent, or addition of a further agent to the therapeutic regime. Alternatively, such an increase or insignificant change may indicate that an alternative treatment should be substituted for the existing treatment.

One of the samples may have been taken after treatment has ceased, such as to monitor for relapse or recurrence of the disease. For example, 1 month, 3 months, 6 months, 9 months, 12 months, 18 months, 2 years, 2-5 years, 3 years, 5 years or longer after treatment has ceased. For example, after the last dosage, or after the patient was determined to be cancer free.

The invention includes the combination of the aspects and preferred features described except where such a combination is clearly impermissible or expressly avoided.

The section headings used herein are for organizational purposes only and are not to be construed as limiting the subject matter described.

All documents mentioned in this text are incorporated herein by reference.

The details of one or more embodiments of the invention are set forth in the accompanying description below including specific details of the best mode contemplated by the inventors for carrying out the invention, by way of example. It will be apparent to one skilled in the art that the present invention may be practiced without limitation to these specific details.

### FIGURES

FIG. 1: PRL-3 can be used as a biomarker for cancer stages. High PRL-3 expresses in 8/10 later stage of patients, while in 1/10 early stage of patients.

FIG. 2: Oncoprotein expression in two normal urine samples and six urine samples from bladder cancer patients. Oncoproteins are commonly detectable in cancer urine samples.

FIG. 3: Lanes 1-2 normal urines, 3-8 cancer urines. PRL-3 can be used as a biomarker for late cancer stages. High PRL-3 expresses in 8/10 later stage of patients, while in 1/10 early stage of patients. Exosome associated PRL3 is present in the urine of bladder cancer patients. Purified exosome fractions from bladder cancer patient urine samples were analysed with antibodies against PRL3, CD63, exosome marker.

FIG. 4: 34 urine samples from lung cancer patients.

FIG. 5: 34 Urine samples from lung cancer patients.

FIG. 6: Oncoprotein expression in lung, urines samples from breast, stomach and nasopharageal carcinoma patients.

FIG. 7: 22 urine samples from lung cancer, cystitis, prostatic Adenocaronima v benign prostatic hyperplasia patients.

FIG. 8: Comparing with normal urine (lane 1), we detect super-strong PRL-3 expressing levels in a large B cell lymphoma patient (lane 2) and low levels in lung cancer urines (lane 3-4).

FIG. 9: PRL-3 negative samples (1,2,3,5) and positive samples (8,9,12,14,16) on a dot blot (left panel) are reconfirmed by a western blot (right panel). We may be able to use dot blot to test large samples at one time.

FIG. 10: Antibody purity tested by SDS PAGE. From the left Ab223, 318, 1992, 1994, reference MoAb and reference rabbit antibody (4%-20% Tris-Glycine Gel)

FIG. 11: Dose response curve for PRL3 prototype device.

FIG. 12: A QC colour chart used to score test line signal; B Prototype test device; C Test device interpretation; D Prototype test device using negative, positive and diluted positive urine samples.

FIG. 13: Intracellular PRL-3 oncoprotein can be secreted into cell culture media and is present in 61% of cancer urines, but not in normal urines. (a) Western blotting of PRL-3 in matched lysates and conditioned culture media of the indicated GC cell lines. CANX, calnexin. (b) Summary of % PRL-3 positivity in urine samples from all cancer patients and normal individuals studied. (c-f) Western blot detection for PRL-3 in the urines of (c) normal individuals and GC patients, (d) nasopharyngeal cancer patients and (e) bladder cancer patients. Representative blots are shown. Mr, relative molecular mass (kDa).

FIG. 14: Effective anti-PRL3 antibody treatment results in a loss of urinary PRL-3, and mechanistically involves intra-tumoral accumulation and recruitment of immune effectors. (a) Western blotting for PRL-3 protein in matched urine and tumor samples from untreated or anti-PRL3 antibody-treated mice harboring PRL-3+ SNU484 or PRL-3− MKN45 orthotopic gastric tumors. Upper panels, excised stomachs at Day 28 (SNU-484) or Day 56 (MKN45). (b) Proposed mechanism of action of anti-PRL3 antibody on PRL-3+ cancer cells. Two forms of secreted PRL-3 antigen are depicted—a soluble, free form, or bound to exosomes.

### EXAMPLES

### Example 1

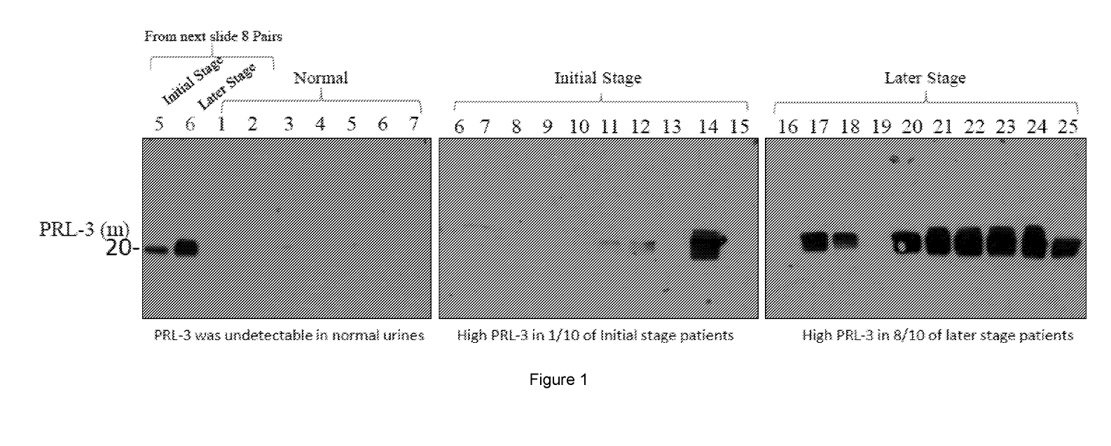

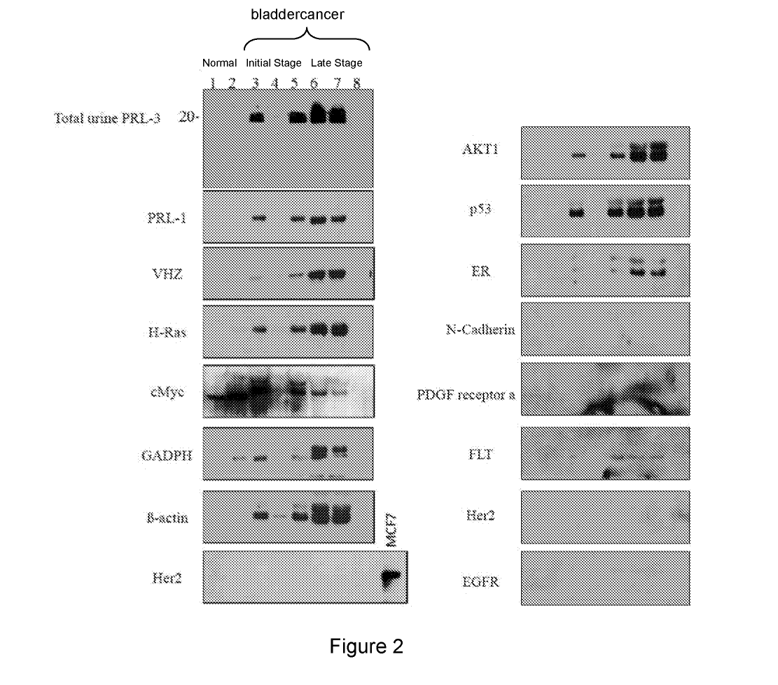

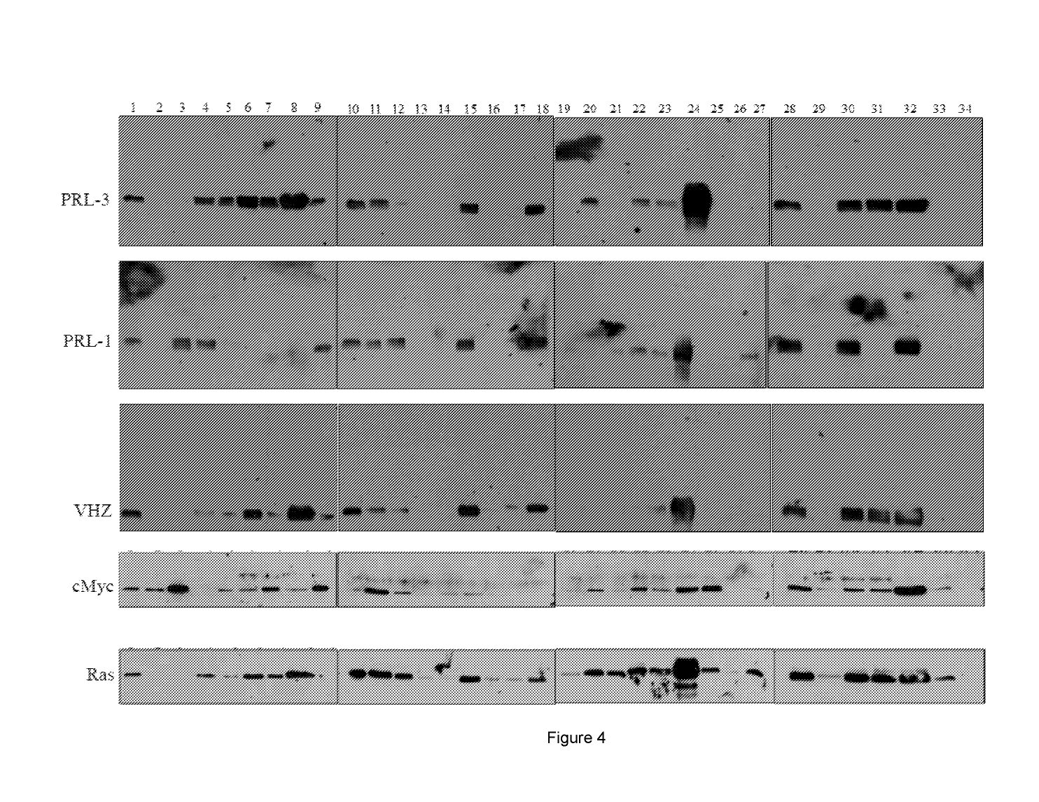

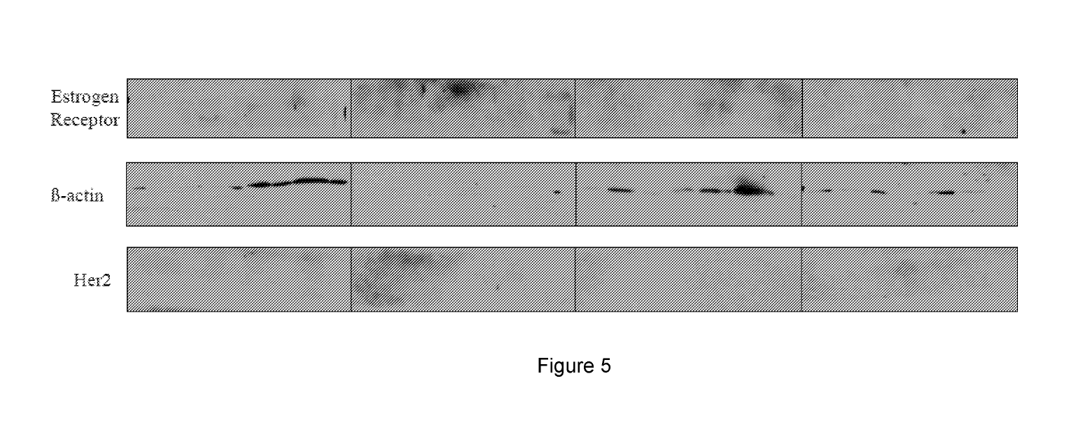

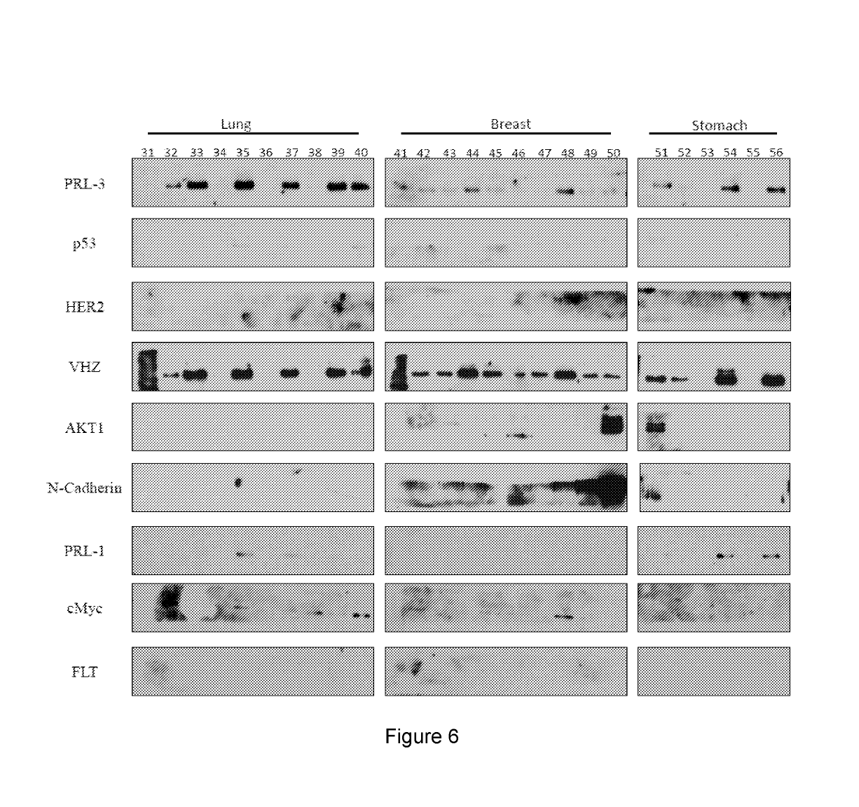

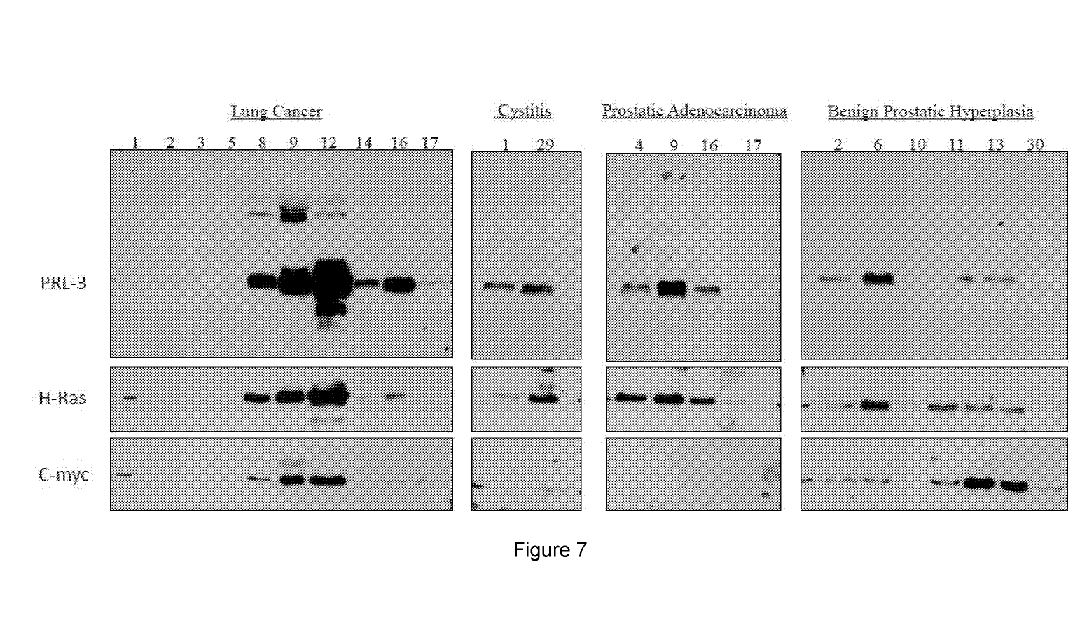

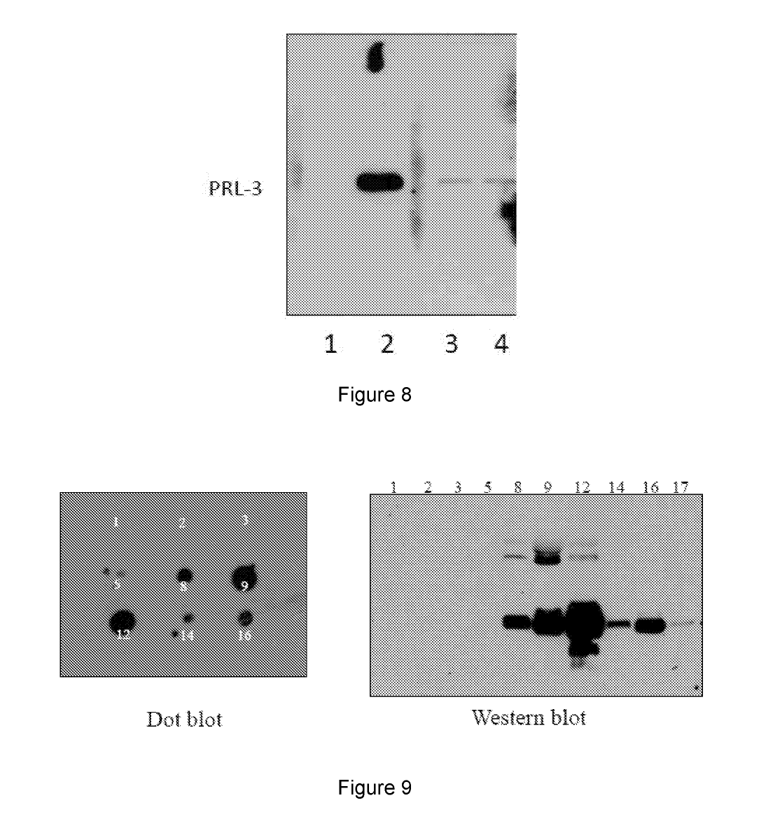

We examined intracellular oncoproteins: PRL-3, PRL-1, VHZ, c-myc, H-ras, AKT-1, p53, Rac1, FAK, Runx1, Estrogen Receptor, PTEN, b-actin, GAPDH from 101 cancer urines (26 bladders, 44 lungs, 10 breasts, 6 Stomach, 15 NPC), and 11 normal urines. We often detect these intracellular oncoproteins in cancer urines. We also detect mutant forms of GAPDH and b-Actin in cancer (but not normal) urines. The results are show in FIGS. 1-8.

We examined extracellular oncoproteins: Her2, N-Cadherin, PDGF receptor (Alpha), FLT-3, p-EGFR from 81 cancer urines (6 bladders, 44 lungs, 10 breasts, 6 Stomach, 15 NPC), and 11 normal urines, we only detected FLT-3 at low levels in 3 out of 6 bladder cancer urines using western blot.

We detected intracellular oncoproteins (much more frequently) than extracellular oncoproteins in cancer urines but not in normal urines. Without wishing to be bound by theory this suggests that extracellular oncoproteins could be more tightly associated with lipid bilayers, therefore, they are more difficult to dissociate from the cell membrane and to travel with exosomes into urine. However, intracellular oncoproteins can be more easily and freely formed and secreted via exosomes to travel into body fluids (blood, urines, saliva). Our findings will facilitate diagnosis for cancer biomarkers used for cancer treatments.

### Example 2

### Exosome Isolation and Kits

This example provides a sample protocol for obtaining proteins from exosomes in a bodily fluid sample. The protocol is represented in FIG. 9.

Protocol: Cat No. EXOAB-KIT-1

If samples are frozen, thaw on ice

Combine 10 ml samples+2 ml ExoQuick-TC i.e, (if 500 ul of samples then 100 ul of ExoQuick-TC)

Mix well by inversion three times

Place at 4° C. for overnight

Centrifuge at 1500*g for 30 minutes or (1.5 rfc)

Remove supernatant, keep Exsosome pellet

Centrifuge at 1500*g for 5 minutes to remove all trace of fluid

Add 100 ul RIPA (radio immunoprecipitation assay) buffer or PBS (according to pellet) to exosome pellet and vortex briefly for 15 seconds.

Place in the room temperature for 20 minutes and take the protein reading at 595 nm.

Add SDS dye and heat up the samples for 5 minutes at 100° C.

Perform the Western blot.

### Example 3

### Evaluation of Prototype Rapid Test Device.

I. Purpose:
1. Study the feasibility to develop a prototype rapid test device for the detection of biomarker PRL3 in patient urine samples.

II. Background

Bio-marker PRL3 was found by Dr. Zeng Qi of IMCB of National University of Singapore in 1998. It had been showed its elevated level in thousands of cancer patient. Dr. Zeng had developed two monoclonal and two rabbit antibodies anti-PRL3. This study intends to evaluate these four antibodies and to investigate the feasibility to use these antibodies to develop an immunochromatographic device using lateral flow technology to distinguish the urine between cancer and normal person.

Rapid test format (lateral flow device) has the advantage of short assay time (only 10-20 minutes to see the test results). Assay also can be performed at the sense or doctor office without special equipment. Dr. Zeng has the interesting to develop such rapid test and contracted AD Consultant (Phoenix, Ariz.) to perform the feasibility study by using these antibodies. The followings are the results of this study.

III. Recommendations:

Based on conclusions in part IV, we recommend:
Set up the specification
   1. Sensitivity, to be determined
   2. Specificity, to be determined
   3. Limit of Detection (LOD): to be determined.
   4. 15 minute assay
   5. Sample type: urine sample
   6. Storage: room temperature.
   7. Expiration date: 24 months after manufacturing
   8. Interference study: to be determined
   9. Submission type: to be determined
   10. Time schedule: to be determined
Use MoAb 223 as captured antibody (for coating) and rabbit anti-PRL3 (1992) as detector (conjugated to gold particles) for rapid test.
Use Nitrocellulose membrane AE99 as solid phase to bind the antibody 223.
Run the clinical samples (50 positives and 100 negatives) to determined specificity and sensitivity of this prototype
If needed, the further improvements will include increasing sensitivity and specificity achieved by using affinity purified rabbit anti-PRL3 antibody.
Feedback for further modification if needed.

IV. Conclusions:
1. MoAb 223 and polyclonal antibody 1992 is the best pair to develop PRL3 rapid test. MoAb 223 was used as detector and polyclonal 1992 as capturer. (see V. 2)
2. The detectability of 10-20 ng PRL3-GST/ml can be achieved in 15 minutes (see V. 3.3)
3. A response curve from 12.5 ng/ml to 4,000 ng/ml was shown in V. 3.1
4. The negative urine and low level sample reproducibility was shown in V. 3.2
5. This prototype can distinguish one positive urine (B) from other 8 negative urine sample (see 3.4)
6. Antibody purification was in V.1
7. Antibody inventory was in VI.

V. Test Results and Discussions:

1. Antibody Purification

Ascites of hybridoma 223 and 318 and antiserum of 1992 and 1993 were received on May 28, 2014. All the four antibodies were purified using Protein. The results were summarized in the Table.

Summary of Protein G Purification of Monoclonal and Polyclonal Antibodies

| | | | Antibody yield | |
|---|---|---|---|---|
| | Volume, | Protein G Purified | from Antiserum | |
| Antiserum/Ascites | ml | Antibody | or Ascites | Lot Number |
| MoAb 318, | 5.1 | 3.42 mg/ml × 3 ml = | 10.26 mg/5.1 mg = | R-1405001 |
| ascites | | 10.26 mg | 2.0 mg/ml | |
| MoAb 223, | 2.3 | 5.96 mg/ml × 0.82 m = | 5.6 mg/2.3 ml = | R-1405002-1 |
| ascites | | 4.88 mg | 2.4 mg/ml | |
| | | 2.06 mg/ml × 0.35 ml = | | R-1405002-2 |
| | | 0.72 mg | | |
| Rabbit anti-PRL3 | 5.5 ml | 4.45 mg/ml × 14.3 ml = | 63.6 mg/5.5 ml = | R-1406002 |
| Antiserum #1992 | | 63.6 mg | 11.56 mg/ml | |
| Rabbit anti-PRL3 | 6 ml | 3.54 mg/ml × 17.8 ml = | 63 mg/6 ml = | R-1406003 |
| Antiserum #1993 | | 63 mg | 10.44 mg/ml | |

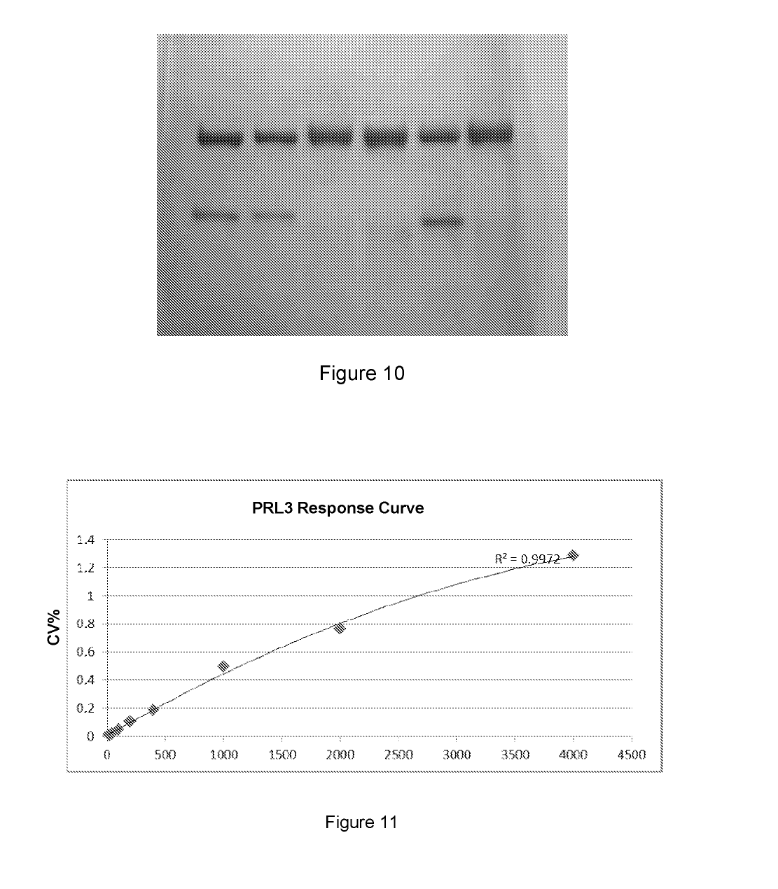

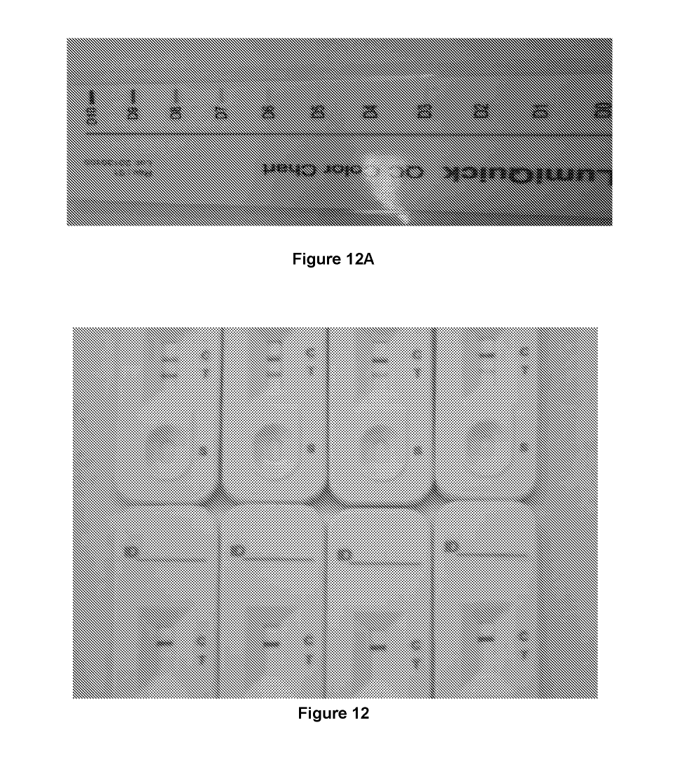

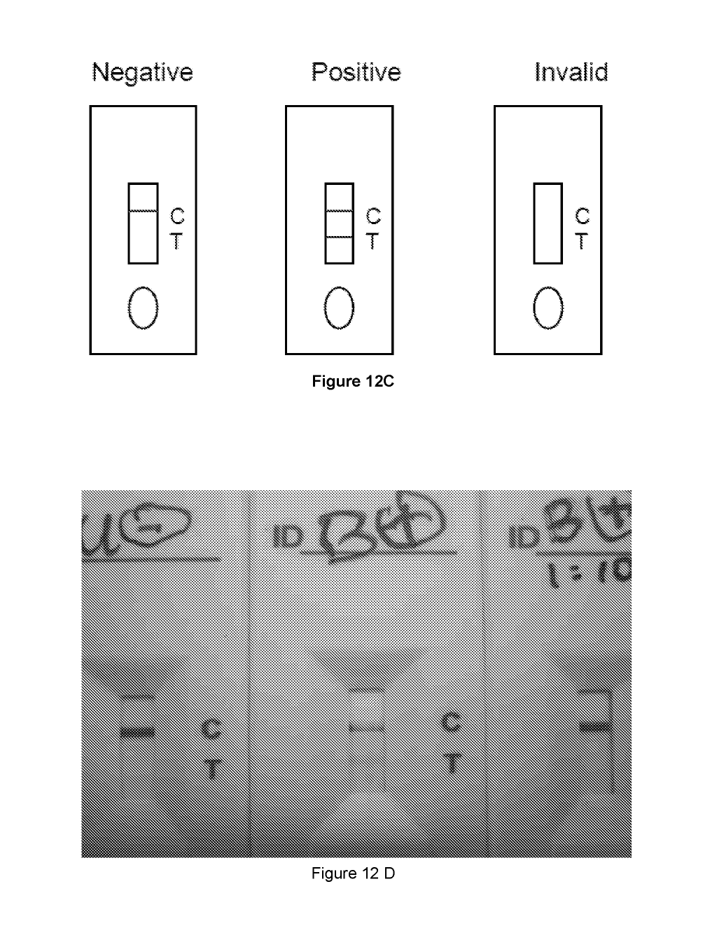

The purity of antibody was tested by SDS-PAGE, see FIG. 10

Discussion:
1. Only light chain (25 kDa) and heavy chain (55 kDa) were seen in PAGE. Purity of antibodies is satisfied.
2. The antibody recovery yield from ascites is 2.0-2.5 mg/ml. This was calculated after dialysis and concentration and based on small amount of ascites (5.1 ml and 2.3 ml).
3. The antibody recovery yield from two rabbit antiserum was 10 to 11 mg/ml respectively.

2. Study on the Best Matching Pair

The Designed Chart for Antibody Pairing

| | | Au-Ab Conjugate | | | |
|---|---|---|---|---|---|
| | | MoAb | MoAb | Rabbit anti- | Rabbit anti- |
| | | 223 | 318 | PRL3 #1992 | PRL3 #1993 |
| Coating | MoAb 223 | 1 | 2 | 3 | 4 |
| Antibody | MoAb 318 | 5 | 6 | 7 | 8 |
| | Rabbit anti- | 9 | 10 | 11 | 12 |
| | PRL3 #1992 | | | | |
| | Rabbit anti- | 13 | 14 | 15 | 16 |
| | PRL3 #1993 | | | | |

Sixteen pairs were studied and the results were listed in the following two Tables.

The Negative Sample Testing Result

| Antibody | Sample | | | | |
|---|---|---|---|---|---|
| Pairing # | buffer 6190 | Urine 1 | Urine 2 | dH₂O | PBS |
| 1 | 0 | 0 | 0 | 0 | 0 |
| 2 | 0 | 0 | 0 | 0 | 0 |
| 3 | 0 | 0 | 0 | 0 | 0 |
| 4 | 0 | 0 | 0 | 0 | 0 |
| 5 | 0 | 0 | 0 | 0 | 0 |
| 6 | 0 | 0 | 0 | 0 | 0 |
| 7 | 0 | 0 | 0 | 0 | 0 |
| 8 | 0 | 0 | 0 | 0 | 0 |
| 9 | 3 | 0 | 0 | 0 | 0 |
| 10 | 0 | 0 | 0 | 0 | 0 |
| 11 | 3 | 0 | 0 | 0 | 2 |
| 12 | 0 | 0 | 0 | 0 | 0 |
| 13 | 0 | 0 | 0 | 0 | 0 |
| 14 | 0 | 0 | 0 | 0 | 0 |
| 15 | 0 | 0 | 3 | 0 | 0 |
| 16 | 0 | 0 | 3 | 0 | 0 |

The Positive Control Testing Result

| | PRL3 GST |
|---|---|
| Antibody Pairing | 100 ng/ml |
| 1 | 2 |
| 3 | 0 |
| 4 | 6 |
| 4 | 3 |
| 5 | 2 |
| 6 | 3 |
| 7 | 3 |
| 8 | 2 |
| 9 | 3 |
| 10 | 0 |
| 11 | 8 |
| 12 | 3 |
| 13 | 2 |
| 14 | 3 |
| 15 | 6 |
| 16 | 6 |

Result and discussion:
2.1 From the positive control testing result, rabbit antibody 1992 showed the highest affinity (pairing 11).
2.2 Based on information of pairing 1-8, 9, 10, 13 and 14, it inferred the affinity of MoAb223 is higher than 318.
2.3 Without affinity purification, polyclonal antibody usually may contain some antibodies came from the infection during animal immunization. The good example is infected by Pseudomonas. After regular purification, this antibody will be introduced into the test line and conjugation in the test device, therefore, to cause false positive if patient sample contains Pseudomonas.
2.4 Due to the reason in 2.3, this study will not consider using polyclonal antibody simultaneously as detector and capturer in the test device.
2.5 Affinity purified rabbit anti-PRL3 can be an option if the sensitivity and specificity are not satisfied after the prototype trial.

3. The Performance of Prototype PRL3 Rapid Test

To perform the PRL3 prototype device on dose response curve and reproducibility, this study used a rapid test reader to quantify the test line signal into digital.

3.1 PRL3 Response Curve

In this study, PRL3 antigen was diluted in sample buffer 6129 (in house). 140 ul of diluted PRL3 was applied to the sample well of the test cassettes (device). After 15 minutes, the test device was read by a rapid test reader. Triplicates were run for each PRL3 level. CV % was calculated for each level.

Result and Discussion:

| Conc. | Run | | | | |
|---|---|---|---|---|---|
| ng/ml | 1 | 2 | 3 | Mean | CV % |
| 12.5 | 0.0087 | 0.0083 | 0.0086 | 0.00857 | 2.4 |
| 25 | 0.0132 | 0.0123 | 0.0127 | 0.012733 | 3.5 |
| 50 | 0.0181 | 0.0186 | 0.0232 | 0.019967 | 14.0 |
| 100 | 0.0425 | 0.0481 | 0.0497 | 0.046767 | 8.0 |
| 200 | 0.1002 | 0.1069 | 0.1054 | 0.104167 | 3.3 |
| 400 | 0.1820 | 0.1925 | 0.2200 | 0.198233 | 9.8 |
| 1000 | 0.4686 | 0.5232 | 0.4998 | 0.4972 | 5.5 |
| 2000 | 0.8349 | 0.7645 | 0.7000 | 0.766467 | 8.8 |
| 4000 | 1.2854 | N/A | N/A | 1.2854 | N/A |

Discussion:

3.1.1 After 2000 ng/ml, the curve became less deep (less sensitivity).

3.1.2 Not hook affection was seen below 4000 ng/ml. The analyst in the curve was PRL3-GST. It is unknown how free PRL3 will be in the hook affection.

3.2 Reproducibility

Assay protocol was same as described in 3.1. 20 runs were repeated for a negative urine sample defined and provided by IMCB. Ten runs were repeated for a low positive PRL3 prepared in sample buffer. CV % was calculated each.

20 Readings of Negative Urine #2

| Reading | | |
|---|---|---|
| Sample number = 10 | Mean | CV % |
| 0.0023, 0.0014, 0.0033, 0.0013, 0.0022 | 0.0027 | 40 |
| 0.0024, 0.0054, 0.0024, 0.0019, 0.0014, | | |
| 0.0030. 0.0026, 0.0025, 0.0018, 0.0041 | | |
| 0.0035, 0.0036, 0.0021, 0.0049, 0.0020 | | |

10 Readings of One Weak Positive Control

| Reading | | |
|---|---|---|
| Sample number = 10 | Mean | CV % |
| 0.0210, 0.0196, 0.0164, 0.0171, 0.0202, | 0.0170 | 14.1 |
| 0.0163, 0.0160, 0.0148, 0.0128, 0.0163 | | |

Discussion

3.2.1 The CV for negative urine is 40%, but the highest and lowest reading was 0.0054 and 0.0014. The threshold for this reader is 0.010 or higher as positive. So this data is acceptable.

3.2.2 The low positive CV is 14.1% and acceptable.

3.3 The Limit of Detection (LOD)

A QC Control Color Chart was used to score test line signal. Score 3 is a borderline and under 3 will regard as negative.

PRL3-GST was diluted in sample buffer at each level shown on the table.

100 ul of dilution was applied to the sample well of test cassette. Score the signal at 15^{th} minute.

| ng/ml | 10,000 | 5,000 | 1,000 | 500 | 100 | 50 | 20 | 10 |
|---|---|---|---|---|---|---|---|---|
| score | 10 | 9 | 8 | 7 | 6 | 5 | 4 | 3 |
| +/− | + | + | + | + | + | + | + | + |

Discussion

3.3.1 The detectability was 10-20 mg T3-GST/ml.

3.2.2 Not hook affection was seen at as high as 10,000 ng/ml.

3.4 Clinical Urine Sample Study

Five negative urine samples and two positive urine samples were received from IMCB. Additional three urine samples were collected from our lab. One of the positive urine samples (L) was used to qualify antibodies. So, only one positive urine sample (Urine B) was used in the final testing using prototype device.

3.4.1 Assay Protocol

1). Bring the urine samples to the room temperature (caution: PRL3 has a different migration behavior on the membrane when it is cool, so bring the sample to room temperature before run the test is very important).

2). Apply 100 ul of urine sample to the test device.

3). Read the result at 15th minute.

4). Test result interpretation:

3.4.2 Assay Results

Sample #1to #5 were negative urine from IMCB, #6 to #8 were from our lab, #9 was positive urine B. Urine B was positive and 1:10 dilution was also seen weak positive.

| #1 | #2 | #3 | #4 | #5 | #6 | #7 | #8 | #9 |
|---|---|---|---|---|---|---|---|---|
| − | − | − | − | − | − | − | − | + |

VI. Antibody Inventory

| | Concentration, | Volume | Total | |
|---|---|---|---|---|
| Antibody/lot number | mg/ml | ml | Mg | |
| MoAb 223/ | | | | |
| R1405002-1 | 5.96 mg/ml | 0.39 ml | 2.3 | mg +) |
| R1405002-2 | 2.06 mg/ml | 0.35 ml | 0.72 | mg |
| | | | 3.0 | mg |
| MoAb 318 | 3.42 mg/ml | 2.8 ml | 9.5 | mg |
| R1405001 | | | | |
| Rabbit anti-PRL3 1992 | 4.45 mg/ml | 14 ml | 62.3 | mg |
| R1406002 | | | | |
| Rabbit anti-PRL3 1993 | 3.54 mg/ml | 17.4 ml | 61.5 | mg |
| R1406003 | | | | |

### Example 4

Unexpectedly, we found secreted PRL-3 oncoprotein in 61% of multiple types of human cancer urines and in all cancer urines derived from PRL-3+ (but not PRL-3−) tumor-bearing mice. Urinary PRL-3 levels were significantly reduced after effective treatment with PRL3-antibody. We propose urinary PRL-3 as a novel diagnostic and a surrogate biomarker for therapeutic response monitoring of PRL3-antibody therapy in multiple cancer types.

Materials and Methods

Western blotting. 200 μg of lysates were resolved in separate wells of 12% SDS-polyacrylamide gels and transferred to nitrocellulose membranes before blocking and probing with the indicated primary antibodies at a 1:1,000 dilution overnight at 4° C. After thorough washing with TBS-T buffer (20 mM Tris pH 7.6, 140 mM NaCl, 0.2% Tween-20), the membrane was incubated with the respective horseradish peroxidase (HRP)-conjugated secondary antibodies at a 1:5,000 dilution for 1 h, washed with TBS-T, and visualized using a chemiluminescent substrate (Pierce).

Antibodies. B cell marker (CD45/CD220, clone RA3-6B2), NK cell marker (CD335/Nkp46, clone 29A1.4), and HSP70 (cat #610607) antibodies were purchased from BD Pharmingen. Calnexin (cat #2679) antibody was purchased from Cell Signaling. CD63 (cat #sc-15363) antibody was purchased from Santa Cruz Biotechnoloy. GAPDH (clone MAB374) antibody was purchased from Millipore.

Urine analysis. Morning urine samples, collected with patients' consent from the National University Hospital of Singapore, were stored at −80° C. prior to analysis. Samples were centrifuged twice at 4,000×g for 20 min at 4° C. to remove insoluble debris prior to analysis. Equal volumes (30 μL) of urine samples were loaded onto gels for western blotting.

Exosome isolation. Exponentially-growing cells at 70-80% confluence were washed twice with PBS prior to incubation with 10 ml of serum-free RMPI-1640 for 24 h. Conditioned medium was centrifuged twice at 300×g for 10 min to remove dead cells and debris. Pre-cleared conditioned medium was subsequently concentrated to ˜500 μL using an ultra-centrifugal 3K filter concentrator (Milipore), and exosomes were extracted from the retentate using the Total Exosome Isolation Reagent (Invitrogen) according to the manufacturer's instructions. For human urine exosome extraction, 1 mL of pre-cleared urine was directly used for exosome extraction as described.

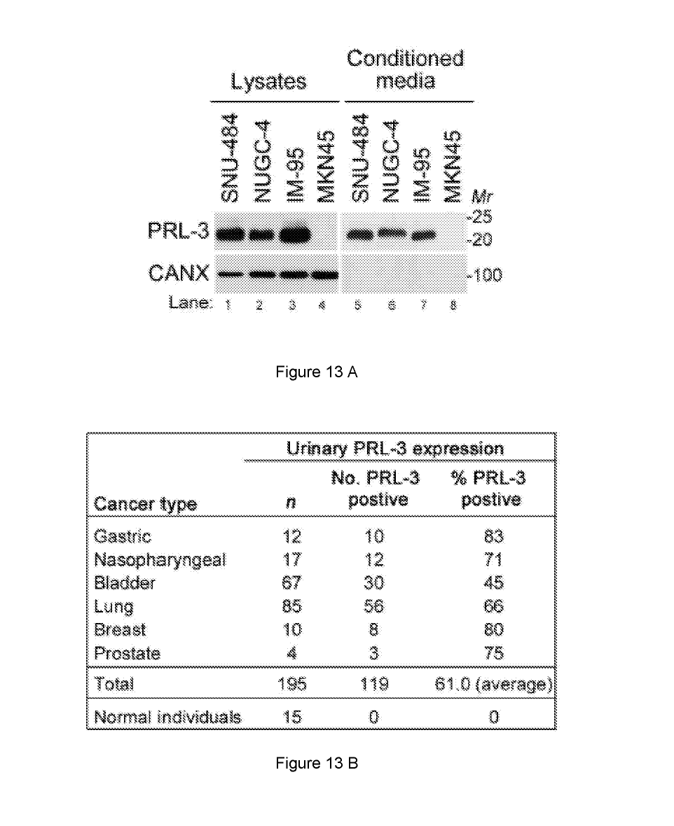

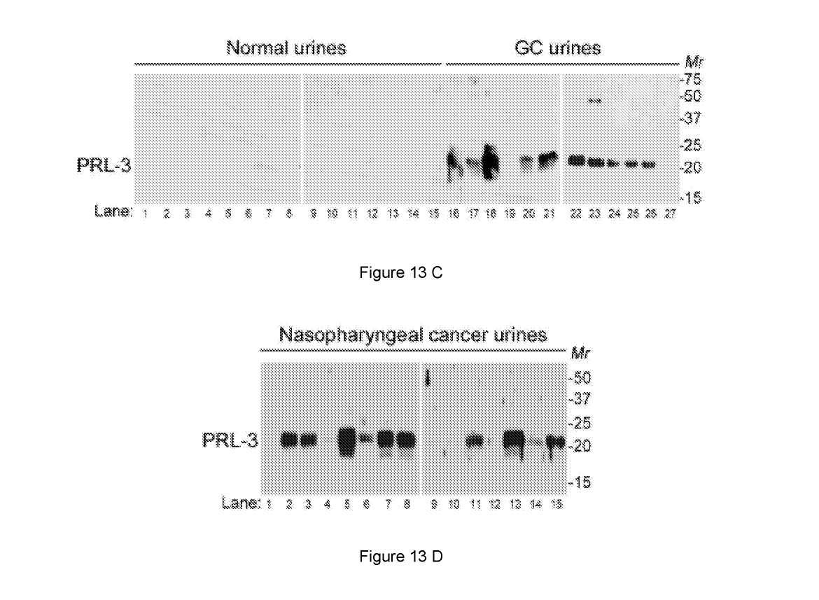

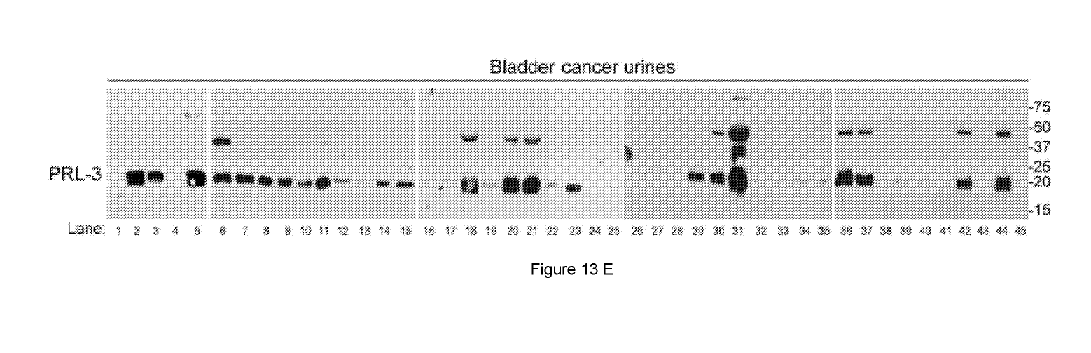

Results Intracellular PRL-3 oncoprotein can be secreted into cell culture media and is present in 61% of cancer urines, but not in normal urines. In 2008, we first reported untraditional immunotherapies against intracellular oncoproteins and demonstrated that PRL-3 antibody could be taken up by tumor cells₂₂. However, it was still unclear how and where antibody recognition of intracellular antigens took place. Herein, we found an unrecognized natural phenomenon that “intracellular” PRL-3 protein could be detected in concentrated culture media from corresponding PRL-3+, but not PRL-3−, cancer cell lines in vitro (FIG. 13a, lanes 5-8). As a control, the ER-localized protein calnexin (CANX) was exclusively found in lysates but not in conditioned media (FIG. 13a), thus ruling out non-specific contamination by dead cells or cellular debris. Since PRL-3 has promising cancer biomarker potential based on microarray and histological studies₆, we proceeded to investigate if “secreted” PRL-3 might have clinical relevance as a biomarker by analyzing urine samples from both healthy individuals and cancer patients.

A total of 15 urine samples from healthy individuals and 195 urine samples from cancer patients were analyzed by western blot to detect PRL-3 protein. Unexpectedly, PRL-3 was readily detected in an average of 61% (119 out of 195) of urine samples from patients with different types of cancer (FIG. 13b), but not in any normal urine samples (FIG. 13c, lanes 1-15). Specifically, urinary PRL-3 protein was detected in 10/12 (83%) of gastric cancer urines (FIG. 13c, lanes 16-27), 12/17 (70%) of nasopharyngeal cancer urines (FIG. 13d), 30/67 (45%) of bladder cancer urines (FIG. 13e), 56/85 (66%) of lung cancer urines (FIG. 13f), 8/10 (80%) of breast cancer urines, and 3/4 (75%) of prostate cancer urines (data not shown). Our results from these 210 urine samples identify PRL-3 as a common cancer-specific urinary protein.

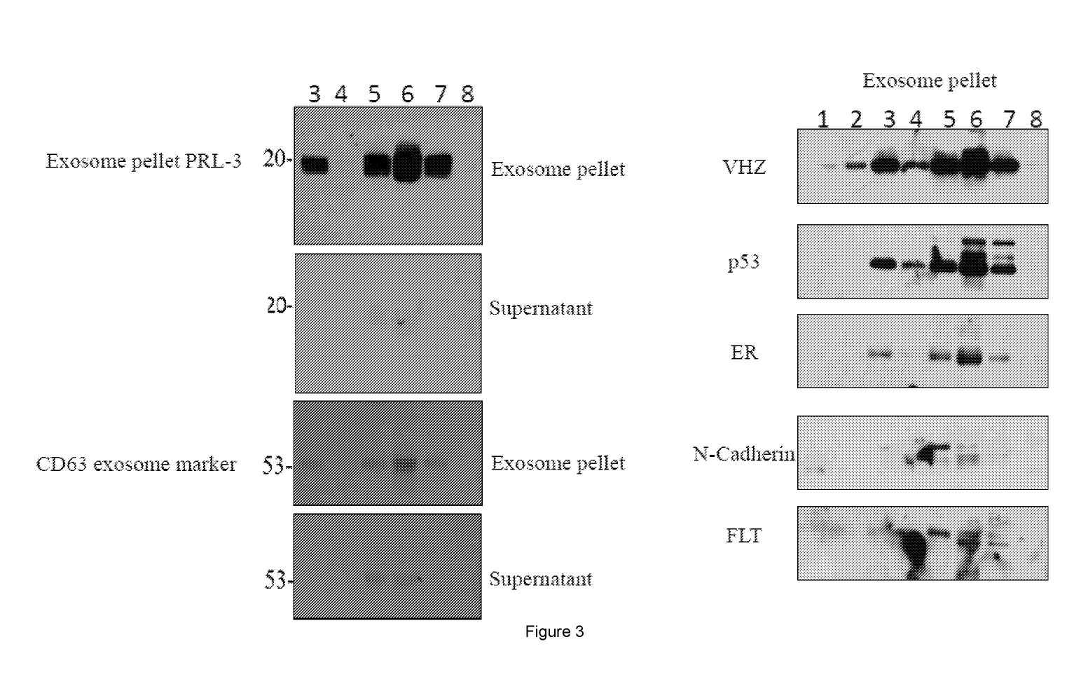

Since PRL-3 protein does not have a sequence peptide for classical secretion via the ER/Golgi pathway, we considered that it might be secreted via non-classical exosome secretion. Exosomes are cell-membrane and/or endosomal-derived vesicles between 50 and 150 nm present in many biological fluids and cell culture media₃₃. To determine if secreted PRL-3 might be exosomal in nature, we performed exosome fractionation of urine samples from patients with different types of cancer, using tetraspanin CD63 as a control exosomal protein₃₄. Surprisingly, we detected exosome-associated PRL-3 exclusively in bladder cancer urines (FIG. 3) but not from other types of cancer urines (data not shown). Since exosomes secreted by epithelial bladder cancer cells bypass physical exclusion limits enforced by glomerular filtration₃₅ and can directly enter the bladder's urine pool, our results indirectly indicate that PRL-3 can be secreted from tumor cells in at least two forms in vivo: 1) As a ˜20 kDa soluble, filterable form presented in multiple types of cancer urines. This ‘free PRL-3’ may leak out into body fluids by tumor necrosis, apoptosis, or tumor cell lysis. Since ‘free PRL-3’ is small enough (˜20 kDa) to pass through the kidney filtration system, which has an estimated cut-off of 70 kDa₃₅, it can readily accumulate in cancer urines. 2) As an exosome-associated form exclusively found in urines of bladder cancer patients. Since circulating exosomes from other cancer tissues (such as gastric, liver, lung) cannot pass through glomerular filtration, only bladder cancer cells with unhindered access to the bladder urinary system could shed such PRL-3-containing exosomes directly into urine. In summary, secreted PRL-3 is a cancer-specific marker comprising of at least two forms—a soluble, ‘free’ form (detectable in urine from various cancer patients), and a larger, exosome-associated form (detectable in urine of bladder cancers patients only).

Urinary PRL-3 is a novel surrogate biomarker for therapeutic response monitoring of anti-PRL3 antibody therapy. Since PRL-3 could be frequently detected in urine samples from cancer patients, we questioned if urinary PRL-3 expression was reflective of the presence of genuine PRL-3+ tumors in vivo. Due to the difficulty in obtaining clinical matched tumor-urine samples to validate this relationship, we instead used PRL-3+ SNU-484 and PRL-3— MKN45 orthotopic gastric mouse models to compare the expression of PRL-3 in matched tumor-urine pairs. In addition, each orthotopic model was sub-divided into 2 groups—mice receiving PBS (untreated), or anti-PRL3 antibody treatment (treated)—to elucidate the relationship between anti-PRL3 antibody therapy and urinary PRL-3 expression.

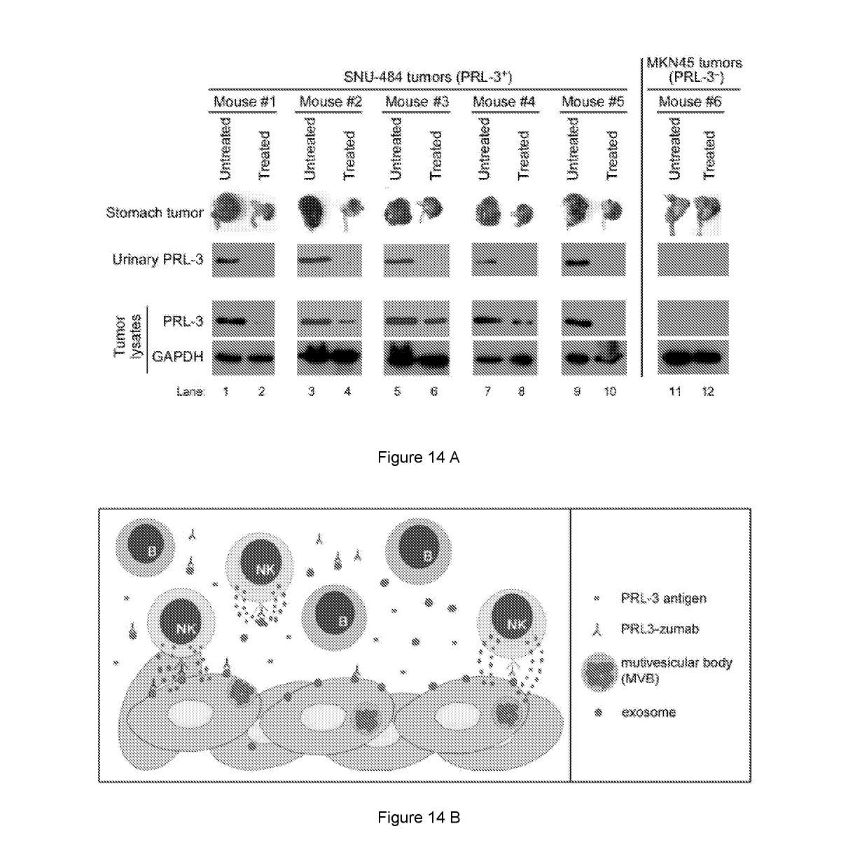

In untreated PRL-3+ SNU-484 tumor-bearing mice, PRL-3 was highly abundant in urine (FIG. 14a, odd lanes 1-9). However, urinary PRL-3 was no longer detectable in all mice after anti-PRL3 antibody treatment, in line with a decrease in intratumoral expression of PRL-3 (FIG. 14a, even lanes 2-10). Importantly, the loss of urinary PRL-3 signal from anti-PRL3 antibody treated mice corresponded with stomach tumor shrinkage in each case (FIG. 14a, upper panels), suggesting that urinary PRL-3 could be useful as a surrogate biomarker of anti-PRL3 antibody therapeutic efficacy. As a control, we did not detect urinary PRL-3 in mice carrying PRL-3-MKN45 orthotopic tumors, regardless of anti-PRL3 antibody therapy (FIG. 14a, lanes 11-12). Thus, urinary PRL-3 is specifically detected in mice carrying PRL-3+ but not PRL-3− cancers, and diminishes upon treatment with anti-PRL3 antibody.

Discussion

This study identifies the biomarker potential of secreted urinary PRL-3 for diagnostic and therapeutic response monitoring. We detected urinary PRL-3 in an average of 61% of multiple human cancer patients.

Although soluble PRL-3 was detected in urines from multiple cancer patients, we detected exosome-associated PRL-3 only in the urines of bladder cancer patients, but not from patients with other malignancies. The likely explanation of this urine is the physical constraint imposed by glomerular filtration, which only allows passage of proteins smaller than 70 kDa from the plasma into the Bowman's capsule for urinary excretion₃₅. Thus soluble, ‘free’ PRL-3 (˜20 kDa), but not exosome-associated PRL-3, remained detectable in the urine from these other cancer types. Nonetheless, the presence of exosome-associated PRL-3 presents an intriguing possibility where budding exosomes from PRL-3+ tumors could serve as anchor points within tumor areas for anti-PRL3 antibody recognition in vivo and initiation of an effector immune response (FIG. 14b).

The close correlation between tumor and urinary PRL-3 expression observed in mouse models suggests that urinary PRL-3 expression could be used as a prospective diagnostic biomarker for PRL-3-targeted cancer therapies (including anti-PRL3 antibody) in a variety of human malignancies. In addition, urinary PRL-3 could also function as a surrogate biomarker, providing a non-invasive, fast, and simple qualitative method for clinicians to infer therapeutic efficacy.

1. A method of determining whether a patient does, or does not, have cancer, comprising determining the presence or absence of an oncoprotein in a sample of bodily fluid from the patient, wherein the presence of the oncoprotein in the sample indicates that the patient has cancer.

2. The method of claim 1 wherein the oncoprotein is an intracellular protein, such as PRL3, PRL1, VHZ, c-myc, H-ras, AKT-1, p53, Rac1, FAK, Runx1, Estrogen Receptor (ER), PTEN, b-actin or GAPDH, preferably PRL3.

3. The method of claim 1 wherein the sample of bodily fluid from the patient is a urine sample.

4. The method of claim 1 wherein the oncoprotein is an extracellular oncoprotein, such as Her2, N-Cadherin, PDGF receptor (alpha), FLT-3 or p-EGFR.

5. The method of any one of the preceding claims further comprising quantifying the level of oncoprotein in the sample.

6. The method of any one of the preceding claims wherein the cancer is an initial or early stage cancer.

7. The method of any one of the preceding claims wherein the cancer is selected from bladder, lung, breast, stomach, nasopharangeal or prostate cancer.

8. The method of any one of the preceding claims wherein the oncoprotein is soluble oncoprotein.

9. A method of determining whether a patient does, or does not, have cancer, the method comprising determining the presence or absence of an oncoprotein in a sample containing exosomes obtained from a bodily fluid from a patient, the sample having been enriched for exosomes, and/or containing substantially only exosomes, wherein preferably the sample is, or is derived from, urine.

10. The method of any one of the preceding claims wherein the oncoprotein is present within the exosomes in the sample.

11. A method of selecting a patient for an anti-cancer therapy, the method comprising detecting the presence of an oncoprotein in a sample of, or derived from, bodily fluid from the patient, and selecting a treatment based on the oncoprotein determined to be present, where the bodily fluid is preferably urine.

12. A lateral flow device comprising one or more anti-oncoprotein antibodies.

13. A kit for detecting oncoprotein in a sample of bodily fluid, the kit optionally including the lateral flow device of claim 12.

14. A method for diagnosing bladder cancer in a patient, the method comprising isolating exosomes from a urine sample from the patient and determining the presence of oncoprotein in the exosomes, wherein the presence of oncoprotein in the exosomes in indicative that the patient has bladder cancer.

15. An anti-cancer therapy antibody for use in the treatment of cancer in a patient, wherein the patient has been selected for treatment by a method comprising determining, in a sample of bodily fluid from the patient, the presence of an oncoprotein.

16. The method, kit or lateral flow device according to any one of the preceding claims wherein the oncoprotein is PRL3.

17. A method of treatment, the method comprising treating a patient selected by a method according to any one of claims 1 to 10.

18. The method of treatment according to claim 17, comprising administering anti-cancer therapy to the patient.

19. The method according to any one of claim 11, 15 or 18 wherein the anti-cancer therapy is an antibody therapy.

20. The method according to claim 19 wherein the antibody therapy is anti-PRL3 antibody therapy.
